SEQUENCE LISTING (12 sequences)

SEQ ID NO: 1 - DNA, 25 bp, Artificial Sequence
  Synthetic oligonucleotide
aaaaaaaaaattgttgatactgttc

SEQ ID NO: 2 - DNA, 25 bp, Artificial Sequence
  Synthetic oligonucleotide
aagaatccaacatttactccaaaaa

SEQ ID NO: 3 - DNA, 30 bp, Artificial Sequence
  Synthetic oligonucleotide
ggagtaaatgttggagaacagtatcaacaa

SEQ ID NO: 4 - DNA, 35 bp, Artificial Sequence
  Synthetic oligonucleotide
atgattaggttgcactcacactattacatctggct

SEQ ID NO: 5 - DNA, 30 bp, Artificial Sequence; identical to SEQ ID NO: 3
  Synthetic oligonucleotide

SEQ ID NO: 6 - DNA, 30 bp, Artificial Sequence
  Synthetic oligonucleotide
ggagaaaatgttggagaacagtatcaacaa

SEQ ID NO: 7 - DNA, 30 bp, Artificial Sequence
  Synthetic oligonucleotide
ggactaaatgttggagaacagtatcaacaa

SEQ ID NO: 8 - DNA, 30 bp, Artificial Sequence
  Synthetic oligonucleotide
ggagaaaaagttggagaacagtatcaacaa

SEQ ID NO: 9 - DNA, 30 bp, Artificial Sequence
  Synthetic oligonucleotide
ggagaaaaagatggagaacagtatcaacaa

SEQ ID NO: 10 - DNA, 35 bp, Artificial Sequence; identical to SEQ ID NO: 4
  Synthetic oligonucleotide

SEQ ID NO: 11 - DNA, 25 bp, Artificial Sequence
  Synthetic oligonucleotide
aagaatccaacatttactccaaaaa

SEQ ID NO: 12 - DNA, 25 bp, Artificial Sequence
  Synthetic oligonucleotide
aaaaaaaaaattgttgatactgttc

(19) United States
(12) Patent Application Publication
(10) Pub. No.: US 2011/0171629 A1
(43) Pub. Date: Jul. 14, 2011

(54) NANOSTRUCTURED DEVICES INCLUDING ANALYTE DETECTORS, AND RELATED METHODS

(75) Inventors: Timothy M. Swager, Newton, MA (US); Yossef Weizmann, Cambridge, MA (US); David M. Chenoweth, Cambridge, MA (US)

(73) Assignee: Massachusetts Institute of Technology, Cambridge, MA (US)

(21) Appl. No.: 12/939,539

(22) Filed: Nov. 4, 2010

Related U.S. Application Data
(60) Provisional application No. 61/258,150, filed on Nov. 4, 2009.

(51) Int. Cl. G01N 33/50; C12Q 1/00; C12Q 1/70; C12Q 1/02; C25B 9/00; B82Y 30/00

(52) U.S. Cl. 435/5; 435/4; 435/29; 436/94; 204/242; 977/742; 977/773; 977/762; 977/750; 977/752

(57) ABSTRACT

The present invention provides compositions and devices comprising nanostructure networks, and related methods. The compositions may exhibit enhanced interaction between nanostructures, providing improved device performance (e.g., improved conductivity). In some embodiments, the devices are capable of interacting with various species to produce an observable signal from the device. In some cases, the compositions and devices may be useful in the determination of analytes, including—biological analytes (e.g., DNA, ebola virus, other infective agents, etc.), small, organic analytes, and the like. The embodiments described herein may exhibit high sensitivity and specificity to analytes and may be capable of analyte detection at femtomolar concentrations (e.g., 10 fM).

### RELATED APPLICATIONS

This application claims priority under 35 U.S.C. §119(e) to co-pending U.S. Provisional Application Ser. No. 61/258,150, filed Nov. 4, 2009, the contents of which application are incorporated herein by reference in its entirety for all purposes.

### STATEMENT REGARDING FEDERALLY SPONSORED RESEARCH OR DEVELOPMENT

This invention was made with the support under the following government contracts: ECCS-0731100 awarded by the National Science Foundation, 1-F32-GM087028-01A1 awarded by the National Institutes of Health, and W911NF-07-D-0004 awarded by the Army Research Office. The government has certain rights in the invention.

### FIELD OF THE INVENTION

The present invention generally relates to compositions and devices for determining analytes, and related methods.

### BACKGROUND OF THE INVENTION

Interfacing carbon nanotubes with biomaterials (DNA, proteins, etc.) for the development of sensitive amplified detection methods often presents a major challenge at the nanotechnology frontier. Amplified DNA detection can be particularly challenging; however sensitive, robust, and economically feasible detection methods for the analysis of genetic disorders or the detection of pathogens and biowarfare agents could have a major impact on human health and safety. Current detection methods have utilized enzymes, nanoparticles, quantum dots, piezoelectric devices, electrochemical approaches, and nano-scale force interactions to address this difficult problem. Alternative methods based on hybrid biomaterial-carbon nanotube (CNT) systems find growing interest in the developing research area of nanotube-based field-effect transistors due to their excellent operating characteristics. Within this area, recent research efforts have focused on the detection of biomaterials using proteins, aptamers, and DNA. CNT network field-effect transistor (CNTNFET) devices for detection of antibodies and DNA have been explored and can offer sensitivity, selectivity, and rapid analysis times coupled with low power and cost benefits.

### SUMMARY OF THE INVENTION

The present invention provides various methods for determining an analyte. In some embodiments, the method comprises exposing, to an environment suspected of containing an analyte, a device comprising a network of nanostructures positioned relative to each other so as to define an area between adjacent nanostructures, wherein the analyte, if present, interacts with at least a portion of the device to produce a species proximate the area between adjacent nanostructures that affects a property of interaction between the nanostructures, thereby producing a detectable change in a property of the network of nanostructures, whereby the detectable change in the property of the network can be determined to determine the analyte.

In some embodiments, the method may comprise exposing, to an environment suspected of containing an analyte, a network of nanostructures positioned relative to each other so as to define an area between adjacent nanostructures, wherein the analyte, if present, interacts with at least a portion of the device to produce a conductive species proximate the area between adjacent nanostructures, thereby generating a determinable signal; and determining the signal.

The present invention also relates to compositions comprising a nanostructure network comprising a plurality of nanostructures and a plurality of linkers attached to the nanostructures, such that the nanostructures and linkers are arranged in a substantially continuous manner along the nanostructure network.

The present invention also relates to compositions comprising a network of nanostructures comprising the formula,

-[A-B—]ₙ— or —[B-A-B]ₙ—,

wherein A is a nanostructure comprising at least two terminal ends; B is a linker; and n is at least 1, wherein the linker is attached to the nanostructure at or near a terminal end of the nanostructure.

The present invention also provide various devices. In some embodiments, the device may be a sensor comprising a composition as described herein. The sensor may further comprise a source of external energy applicable to the composition to generate a determinable signal, and a detector positioned to detect the signal. In some embodiments, the device comprises a composition as describer herein; at least two electrodes in electrochemical communication with the composition; and optionally, a conductive material in electrochemical communication with the composition and at least two electrodes.

The present invention also provides methods for synthesizing a nanostructure network comprising reacting a nanostructure species comprising at least two reactive sites with a linker species comprising at least two reactive sites to produce a nanostructure network comprising nanostructure species and linker species arranged in a substantially continuous manner along the nanostructure network.

### BRIEF DESCRIPTION OF THE DRAWINGS

FIG. 1A shows schematic representations of a sensing scheme for DNA-CNT disrupted nanowire-network devices, including sensing and silver development using (I) a Au nanoparticle probe and (II) peroxidase enzyme (HRP) probe II.

FIG. 1B depicts a DNA sensing gap between surfactant-shielded nanotubes showing the DNA sequence composition for the capture, target, probe, and control DNA strands.

FIG. 2 shows (a) synthesis of a nanostructure network, (b) fabrication of a device including a nanostructure network, (c) an atomic force microscopy (AFM) image of a nanotube network with DNA analyte and Au nanoparticles present at the junction between adjacent nanostructures, and (d) confocal Raman spectra of the nanotube network in (c), wherein the presence of nanotubes is verified.

FIG. 3A shows a horseradish peroxidase (HRP) crystal structure (PDB 1hch) with potential nucleophilic residues for DNA conjugation mapped on the surface, and a corresponding table of calculated solvent accessible surface areas (SAS) for each residue.

FIG. 3B shows the synthesis of a DNA-conjugated HRP molecule.

FIG. 3C shows UV-Vis spectra for (I) native HRP, (II) DNA, and (III) DNA-conjugated HRP.

FIG. 3D shows a graph of enzyme activity assay for native (I) HRP and (II) DNA-conjugated HRP.

FIG. 4 shows (a) relative resistance data from studies using Au nanoparticle probes as a catalyst for silver deposition and (b) relative resistance data from studies using HRP probe as a biocatalyst for silver deposition.

FIG. 5A shows confocal Raman data for device junctions (I) before silver deposition and after silver deposition, using (II) HRP or (III) Au nanoparticle silver development probes using laser wavelength of 784.4 nm (Elaser=1.58 eV).

FIG. 5B shows confocal Raman data for device junctions (I) before silver deposition and after silver deposition, using (II) HRP or (III) Au nanoparticle silver development probes using laser wavelength of 632.8 nm (Elaser=1.96 eV).

FIG. 5C shows confocal Raman data for device junctions (I) before silver deposition and after silver deposition, using (II) HRP or (III) Au nanoparticle silver development probes using laser wavelength of 532.2 nm (Elaser=2.33 eV).

FIG. 6 shows AFM images of (a) oxidized, shielded carbon nanotubes after sonication, (b) a DNA-conjugated CNTs with terminal connections mediated by DNA junctions, (c) another DNA-conjugated CNTs with terminal connections mediated by DNA junctions, and (d) DNA-conjugated CNTs with bound Au nanoparticle probes and target DNA (1 nM).

FIG. 7 shows a histogram of the distribution of carbon nanotube lengths after oxidation procedure (Mean=196, Median=175, n=100).

FIG. 8 shows the recognition domain of a DNA-conjugated CNT network, according to one embodiment.

FIG. 9 shows a graph of conductimetric response data from a concentration dependent DNA-detection study.

FIG. 10 shows normalized conductimetric response data for a DNA-conjugated CNT network for single, double, and triple mismatch oligonucleotide sequences compared to a match sequence.

FIG. 11A shows confocal Raman data for junctions between adjacent nanotubes in a DNA-conjugated CNT network before (bottom curve=no probe) and after silver deposition (top curve=target device, middle curve=mismatch control device).

FIG. 11B shows SEM images of a DNA-conjugated CNT network.

FIG. 12 shows the synthesis of various DNA-conjugated CNT networks.

FIG. 13 shows the manufacture of a DNA-conjugated nanostructure device using directed assembly and a functionalized substrate.

Other aspects, embodiments and features of the invention will become apparent from the following detailed description when considered in conjunction with the accompanying drawings. The accompanying figures are schematic and are not intended to be drawn to scale. For purposes of clarity, not every component is labeled in every figure, nor is every component of each embodiment of the invention shown where illustration is not necessary to allow those of ordinary skill in the art to understand the invention. All patent applications and patents incorporated herein by reference are incorporated by reference in their entirety. In case of conflict, the present specification, including definitions, will control.

### DETAILED DESCRIPTION

Compositions and devices including nanostructure networks are provided, including those capable of interacting with various species to produce an observable signal. In some cases, the compositions and devices may be useful in the determination of analytes, including biological analytes (e.g., DNA, ebola virus, other infective agents, etc.), small, organic analytes, and the like. Embodiments described herein may exhibit high sensitivity and specificity to analytes and may be capable of analyte detection at femtomolar concentrations (e.g., 10 fM). Compositions, devices, and methods described herein may provide a rapid, sensitive, operationally simple, and cost effective means for the detection of target analytes and may be used in various biological, chemical, medical, health, bio-diagnostic sensory, and security applications. In some embodiments, nanostructure materials having improved electrochemical properties (e.g., low resistance) are provided and may be useful in various electrochemical applications, including nano-circuits and electrode assemblies. Additionally, methods for synthesizing nanostructure networks are described, including methods for regioselective functionalization of nanostructures and assembly of nanostructure networks.

An advantageous feature of some embodiments described herein is the ability to functionalize nanostructures regioselectively in order to synthesize various monomeric species for use in nanostructure networks. Such nanostructure species may be readily modified to produce a nanostructure network having desired properties, as described more fully below. In some cases, nanostructure networks having enhanced electrochemical communication are provided. For example, the resistance between adjacent nanostructures within a network may be reduced using compositions and methods described herein, resulting in improved device performance and sensitivity.

Some embodiments of the invention provide methods for determination of an analyte. The analyte may be determined by monitoring, for example, a change in a signal (e.g., an electrical signal) of a material present within the device, upon exposure to an analyte. In some cases, the change in signal may be associated with an interaction between the device and the analyte, a chemical reaction within the device, or a change in a conductive state of a component of the device. The signal may comprise an electrical, or other property of the device, as described further below. For example, the method may involve use of a component having a conductivity, where the conductivity of the component is affected by (e.g., responsive to) an analyte. Such as signal can be read-out by a simple, low power and low current circuit, without need for a bulky apparatus. In other embodiments, the capacitance of a component is affected by the analyte, where the change in capacitance may be determined without need for a complete circuit.

Some embodiments of the invention may also provide devices (e.g., sensors), or systems comprising a plurality of devices, for determination of analytes. In some embodiments, the device may include a material that may be affected by interaction with an analyte, generating a determinable signal. In some cases, the device may comprise a material that is responsive to a biological analyte (e.g., nucleic acid molecule). The device, or portion thereof, may interact with an analyte such that a chemical or biological event (e.g., chemical reaction, biological binding, change in conductivity or capacitance state) occurs, giving rise to a determinable signal or change in signal. The device may comprise a material that is capable of undergoing a change in one or more properties upon exposure to an analyte. For example, the device may include a conductive, semiconductive, or semimetallic material having electrical properties that may be affected by the presence of the analyte. In some cases, an optical, volumetric, or dimensional property of the device may be affected by the analyte. The devices described herein may be designed and fabricated to determine one or more analytes.

In some embodiments, the device may be a sensor comprising any of the compositions described herein. The sensor may also include a source of external energy applicable to the composition to generate a determinable signal from the composition, as well as a detector positioned to detect the signal. The source of external energy may be thermal, electric, magnetic, optical, acoustic, electromagnetic, mechanical or the like. In some cases, the source of external energy may be electromagnetic radiation, such as ultraviolet light or visible light. In some cases, the source of external energy may be electric. In an illustrative embodiment, the device may comprise a plurality of conductive components positioned within the device such that, in the presence of an analyte, a property of interaction between the conductive components may be affected, thereby indicating the presence and/or amount of an analyte. The property of interaction may include, for example, the morphology, orientation, electrochemical state, polymerization state, distance between adjacent components, or other property, of the conductive components. For example, the analyte may interact with one or more portions of the device to affect the electrochemical state (e.g., conductivity, capacitance) of the conductive components. In some embodiments, the presence of an analyte may cause generation of a species that may affect the interaction between the conductive components, as described more fully below.

Compositions and devices described herein may also be useful in various electrochemical applications, as described more fully below. For example, the compositions may be incorporated into electrodes to create nano-circuits. In some embodiments, the compositions may improve the electrochemical properties of conductive components (e.g., nanostructures) within the devices, including the electrochemical communication between adjacent conductive components.

In some embodiments, the composition or device may comprise a conductive, semiconductive, semimetallic species, or other species capable of transporting charge to create a conductive pathway. The conductive, semiconductive, or semimetallic species may include inorganic materials (e.g., metals, alloys, semiconductors), organic materials (e.g., polymer materials), organometallic materials, and/or combinations thereof. In some cases, the material may include a plurality of nanostructures (e.g., nanotubes, nanowires, nanoribbons, nanoparticles, etc.). The nanostructures may be selected to exhibit, for example, high charge mobilities. In some cases, mixtures or assemblies of nanostructures may be utilized. Some embodiments may involve the use of nanotubes, such as single-walled carbon nanotubes (SWCNTs) and/or multi-walled carbon nanotubes (MWCNTs), which can display relatively high charge mobilities (e.g., 100,000 cm²/Vs for SWCNTs).

In some embodiments, the composition or device comprises a network of nanostructures, in which the nanostructures may be positioned relative to each other so as to define an area between adjacent nanostructures. For example, the nanostructures may be arranged such that a linear network is formed, where adjacent nanostructures are separated by a certain distance along the axis of the network. As used herein, a “linear” network of nanostructures refers to a sequence of nanostructures oriented such that terminal ends of adjacent nanostructures are facing one another. That is, the minimal distance between adjacent (e.g., adjacent and attached) nanostructures in a linear network is represented by the terminal ends of the adjacent nanostructures. In some cases, the long axis of linear network may form a substantially straight line. In some cases, the long axis of linear network may adopt a curved line over a distance. In another set of embodiments, the nanostructures may be arranged such that a branched network is formed. As used herein, a “branched” network of nanostructures refers to a sequence of nanostructures oriented such that terminal ends of adjacent nanostructures are facing one another, wherein the long axis of the network may include one or more “arms” or “branches.”

In some embodiments, adjacent nanostructures may be attached to one another via one or more linkers which may determine the distance between adjacent nanostructures. For example, the device may comprise a nanostructure network comprising a plurality of nanostructures and a plurality of linkers attached to the nanostructures, such that the nanostructures and linkers are arranged in a substantially continuous manner along the nanostructure network. In some cases, the nanostructures and linkers are arranged in an alternating manner along the nanostructure network. In some cases, the nanostructures are attached to the linkers via sites at or near terminal ends of the nanostructures. In some cases, the nanostructures and linkers are attached via covalent bonds. In some cases, the nanostructures and linkers are attached via non-covalent bonds, including hydrogen bonds (e.g., DNA hybridization).

In one set of embodiments, the network of nanostructures comprises the formula,

-[A-B—]ₙ— or —[B-A-B]ₙ—,

wherein A is a nanostructure comprising at least two terminal ends; B is a linker; and n is at least 1, wherein the linker is attached to the nanostructure at or near a terminal end of the nanostructure. As described herein, the linker may interact with the nanostructure species through non-covalent interactions (e.g., DNA hybridization) or covalent interactions. In some cases, n is at least 10, at least 100, at least 1000, at least 10,000, at least 100,000, or greater. It should be understood that other nanostructure-linker configurations are also possible, such as alternating pairs of nanostructures and linkers, nanostructures randomly interspersed between series of linkers, or linkers randomly interspersed between series of nanostructures. In some embodiments, the network of nanostructures may further comprise at least one site capable of interacting with (e.g., binding to, hybridizing with, forming a covalent bond with, etc.) an analyte.

In some embodiments, the nanostructure species may be a nanotube, nanorod, nanoribbon, nanowire, nanoparticle, or the like. In some embodiments, the nanostructures are nanotubes, such as carbon nanotubes. The nanostructures may be single-walled carbon nanotubes or multi-walled carbon nanotubes. In some cases, the nanostructures may be conductive nanotubes, semiconductive nanotubes, or metallic nanotubes. In some embodiments, the nanostructures are metal nanowires.

The nanostructures may also include at least two functional groups or reactive sites capable of forming a bond (e.g., covalent bond, non-covalent bond) with the linker species. The groups or sites may be positioned at or near terminal ends of the nanostructures. In some cases, the nanostructures may comprise biological molecules positioned at or near terminal ends of the nanostructures. The biological molecule may be a nucleic acid molecule, peptide, protein, glycoprotein, enzyme, DNAzyme, aptamer, hormone, antibody, antigen, cell, bacteria, virus, carbohydrate, or the like. In one set of embodiments, the nanostructures may be nanotubes comprising carboxylic acid groups, alcohols, amides, acid chlorides, azides, alkynes, maleimides, biotin groups, peptide chains, succinate esters, or the like, positioned at or near terminal ends of the nanostructures. In another set of embodiments, the nanostructures may comprise single-strand DNA groups positioned at or near terminal ends of the nanostructures.

In some embodiments, the nanostructures may also comprise sites capable of interacting with an analyte. The sites may be positioned at or near the terminal ends of the nanostructures (or in regions between adjacent nanostructures within the network), or at other locations of the nanostructures. For example, the nanostructure may be a nanotube, with sites capable of interacting with an analyte positioned at or near the terminal ends of the nanotube, or along the sidewalls of the nanotube.

The linker may be any species capable of forming bonds to at least two nanostructures, i.e., may comprise at least two functional groups or reactive sites capable of forming a bond (e.g., covalent bond, non-covalent bond) with the nanostructure species. In some cases, the functional group may be an amine, a hydroxyl group, a carbonyl group, an olefin, or the like. In some embodiments, the linker may comprise a biological molecule, such as a nucleic acid molecule (e.g., DNA). In some embodiments, the linker may be capable of binding to, or hybridizing with, at least a portion of the nanostructure species. In some embodiments, the linker may be selected to have sufficient size in order to provide the desired spacing between adjacent nanostructures. The linker may also be selected to be capable of binding to, hybridizing with, or otherwise interacting with a target analyte. In some embodiments, the linker may comprise various binding sites (e.g., receptors) for a particular analyte. In some cases, the linker may comprise a group capable of forming a secondary structure (e.g., double-stranded helix) with a nanostructure species and/or an analyte.

In some cases, the linker comprises a biological molecule. Examples of biological molecules or “biomolecules” include nucleic acid molecules, peptides, proteins, glycoproteins, enzymes, DNAzymes, aptamers, hormones, antibodies, antigens, cells, bacteria, viruses, carbohydrates, and the like. In some embodiments, the linker comprises DNA. In some embodiments, the linker comprises an antibody. In some cases, the linker may comprise a polymer. The polymer may be substituted with one or more binding sites, such as a biological molecule capable of interacting with an analyte. For example, the linker may be a polymer substituted with a plurality of carbohydrates, which may serve as a multivalent receptor for biological analytes, such as E. coli. In some embodiments, the polymer may be a conducting polymer, such as polythiophene, polypyrrole, or the like. The linker may also comprise a nanoparticle, including metal nanoparticles (e.g., gold nanoparticles).

Some embodiments involve use of a linker substituted with at least one biological molecule. In one set of embodiments, the linker species be substituted with at least two single-strand DNA (ssDNA) groups. That is, the linker species may have the following formula,

ssDNA-X-ssDNA,

where X is a biological molecule, a nanostructure (e.g., nanotube), a polymer, a nanoparticle, small molecule, or any combination thereof. The single-strand DNA groups may be selected to be complementary to single-strand DNA groups on a nanostructure species, such that the nanostructures and linkers may assemble into a network via DNA hybridization. In illustrative embodiment, the linker species may be a nanotube substituted with single-strand DNA groups complementary to single-strand DNA groups on a nanostructure species, and positioned at or near the terminal ends of the nanotube. In another embodiment, the linker may be nanoparticle (e.g., a gold nanoparticle) functionalized with single-strand DNA groups that are complementary to single-strand DNA groups on a nanostructure species. In some embodiments, X may include a site capable of interacting with an analyte.

In one set of embodiments, the network of nanostructures may comprise nanotubes (e.g., carbon nanotubes) and nucleic acid molecules (e.g., DNA) arranged in an alternating manner along the nanostructure network.

Devices described herein may comprise one or more additional components that may facilitate and/or enhance the electrochemical properties of nanostructure network and/or the responsiveness of the device to the analyte. In some cases, the device includes at least one component that enhances interaction between the nanostructure network and an analyte, improves sensitivity for a particular analyte, or otherwise improves performance (e.g., improves conductivity, capacitance, resistance, etc.) of the device. In embodiments where the device is capable of determining an analyte, the component may be positioned within the device or in sufficient contact with the device such that interaction of an analyte with the nanostructure network and/or component causes, enhances, or otherwise facilitates a determinable change in signal upon exposure to an analyte. In some embodiments, the component(s) may be positioned within the device to enhance electrochemical communication between conductive components (e.g., nanostructures) present within the device. In some embodiments, the device may be contacted with or immersed within a solution comprising the components. Some embodiments may involve the use of one or more probes and/or precursor moieties that interact with an analyte and/or cause a change in a property of interaction between nanostructures. For example, the probe and/or precursor moiety may be capable of affecting electrochemical communication between nanostructures. In some embodiments, the device comprises one type of probe. In some embodiments, the device comprises several different types of probes. The probe may be attached to the nanostructure network, or may otherwise be positioned in contact with the nanostructure network.

The probe may comprise any moiety capable of producing a species that can affect a property of interaction between the nanostructures. For example, the probe may comprise a moiety capable of oxidizing, reducing, polymerizing, or otherwise causing conversion of (e.g., chemically reacting) a component to form a species affecting the interaction between nanostructures. In some cases, the device may be in contact with a precursor moiety that is capable of being converted, by the probe, to a conductive species. The precursor moiety may be a metal, metal ion, or a metal-containing group such that, upon interaction with the probe, the precursor moiety is reduced or oxidized. In some embodiments, the precursor moiety is a monomer such that upon, interaction with the probe, the precursor moiety is polymerized. The probe may further comprise at least one group capable of interacting with at least a portion of the analyte via, for example, covalent bonding, non-covalent bonding (e.g., hydrogen bonding, ionic bonding, dative bonding), or a binding event (e.g., biological binding). The group may be a nucleic acid molecule, peptide, protein, glycoprotein, enzyme, DNAzyme, aptamer, hormone, antibody, antigen, cell, bacteria, virus, carbohydrate, and the like. In some embodiments, the probe comprises a nucleic acid molecule and is capable of interacting with a portion of the analyte via nucleic acid hybridization (e.g., DNA hybridization).

The probe may be capable of affecting the interaction between nanostructures by producing a species (e.g., conductive species) capable of affecting the interaction between nanostructures in a location proximate the nanostructure network. In some embodiments, the probe may be capable of producing the species in highly localized manner at a particular location of the nanostructure network, as described more fully below. For example, a conductive species may be formed proximate the area between adjacent nanostructures so as to create or increase electrochemical communication between adjacent nanostructures, leading to improved conductivity and/or amplified conductimetric detection of analytes.

In some cases, the probe comprises a nucleic acid molecule, aptamer, enzyme, or other biological molecule, or a metal (e.g., metal nanoparticle) or metal-containing compound. For example, the probe may comprise a metal nanoparticle, such as a catalytic gold nanoparticle, capable of oxidizing or reducing a metal-containing species. In some embodiments, the probe comprises an enzyme capable of oxidizing or reducing a metal-containing species. For example, the enzyme (e.g., peroxidase enzymes such as horseradish peroxidase) may be capable of reducing silver ions to silver metal, providing a means for silver deposition at disrupted nanotube network connections. Other enzymes suitable for use as probes include glucose oxidase, alkaline phosphatase, DNAzymes, and the like.

Exposure of the nanostructure network to a probe and/or precursor moiety may allow for formation of a conductive material in or near at least some of the areas between adjacent nanostructures, establishing electrical contacts between nanostructures and enhancing electrochemical communication between nanostructures. The nanostructure network may interact with the probe, precursor moiety, and other species involved in forming the conductive material via various interactions, including covalent bonding, non-covalent bonding (e.g., biological binding), and the like. In some embodiments, a conductive material may be formed in or near substantially all of the areas between adjacent nanostructures to form a substantially continuous conductive pathway through the network. In some embodiments, the conductive material may be formed in or near some of the areas between adjacent nanostructures, while other areas between adjacent nanostructures may not comprise the conductive material. In such cases, areas which may not comprise a conductive material may include, for example, a group or a binding site for determination of an analyte,

In some cases, the conductive material may be a metal (e.g., silver metal), metal ion, or other metal-containing species. For example, the network may comprise a plurality of single-strand DNA groups positioned between adjacent nanostructures, and the network may be placed in contact with a solution of silver ions and an enzyme functionalized with single-strand DNA groups complementary to those of the network. The enzyme may interact with the network via DNA hybridization and may reduce the silver ions to silver metal in the area between adjacent nanostructures. In another example, the network may comprise an alternating arrangement of nanostructures and metal nanoparticles (e.g., gold nanoparticles), and may be placed in contact with (e.g., immersed within) a solution of silver ions. The gold nanoparticles may then be used to reduce the silver ions to silver metal, which may be formed in the area between adjacent nanostructures.

In some embodiments, formation of a conductive material in the presence of a probe and/or precursor moiety may be useful in the determination of analytes, as described more fully below. For example, the probe may be selected to produce a conductive species in a location proximate the nanostructure network upon exposure to a particular analyte. In one set of embodiments, the device may be in contact with a probe comprising glucose oxidase, which is capable of converting glucose to peroxide, and a precursor moiety comprising a monomeric species such as aniline. In the presence of analyte, the probe may convert glucose to peroxide, which may then initiate polymerization of the monomeric species at the junction between adjacent nanostructures. Thus, a polymer (e.g., polyaniline) may be deposited at the spaces between nanostructures to create an uninterrupted conductive pathway comprising nanostructures and the polymer. Formation of a conductive material between nanostructures may, in some cases, advantageously reduce the resistance between adjacent nanostructures within the network, allowing for the formation of highly sensitive devices for determination of analytes. That is, reducing the overall resistance of a device prior to exposure to an analyte may increase the sensitivity of the device, as analyte-induced changes in resistance can be more readily determined.

In some cases, the nanostructure network may comprise a first set of regions between adjacent nanostructures, wherein the regions comprise groups capable of interacting with a probe and/or precursor moiety such that a conductive material (e.g., silver) may be formed in the regions, and a second set of regions between adjacent nanostructures, wherein the regions comprise groups capable of interacting with an analyte. In some embodiments, a conductive material may be selectively formed, as described herein, at the first set of regions but not the second set of regions, to produce a network having a conductive pathway interrupted by sites to which an analyte may bind. In such cases, an analyte may bind to the network at the second set of regions, causing formation of a conductive material at the second set of regions, thereby forming a substantially continuous conductive pathway through the network and producing a change in a determinable signal of the network.

For example, a network may include a first set of DNA groups positioned between adjacent nanostructures, wherein the DNA groups may be selected to interact with a probe and/or precursor moiety to cause formation of silver metal at the first set of DNA groups, and a second set of DNA groups selected to interact with an analyte. The network may be treated with the probe and precursor moiety such that, prior to exposure to an analyte, the network may include a conductive pathway interrupted by sites capable of interacting with an analyte. Upon exposure to an analyte, the analyte may interact with the second set of DNA groups, causing formation of silver metal or another conductive material, producing a completed circuit throughout the network and giving rise to a determinable change in signal (e.g., change in conductivity, resistance, etc.) to determine the presence and/or quantity of analyte. As described herein, the number of binding sites for an analyte may be modulated to increase the sensitivity of the network for an analyte. In some cases, the network may be designed to include a reduced number of analyte binding sites (or number of interruptions to the conductive pathway), such that a reduced number of analyte-binding events may be needed to form a complete circuit throughout the network.

In other embodiments, the network may comprise a conductive material formed on substantially all regions between adjacent nanostructures. The network may further comprise sites for binding and/or interacting with analytes arranged, for example, along the sidewalls of nanostructures, such as single-walled carbon nanotubes. Those of ordinary skill in the art would be able to select and/or synthesize nanostructures having binding sites for analytes positioned at various locations of a nanostructure. Examples of methods for functionalizing nanostructures are described in International Publication No. WO2008/133779 and International Publication No. WO/2009/136978, the contents of which applications are incorporated herein by reference in their entirety for all purposes.

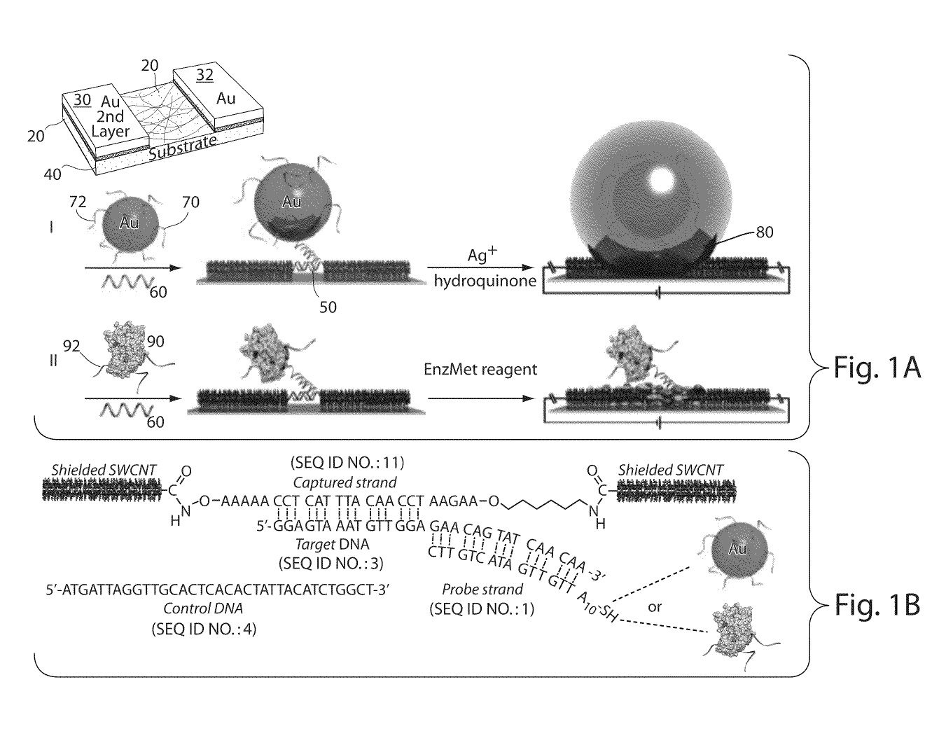

The device may further comprise a first electrode and a second electrode arranged in electrochemical communication with the network of nanostructures, where current flow between the electrodes is affected by the nanostructure network. FIG. 1A shows an illustrative embodiment of device 10, prior to exposure to an analyte, where nanostructure network 20 is formed on substrate 40 and is in electrochemical communication with electrodes 30 and 32. Nanostructure network 20 comprises a linear network of nanostructures, with terminal ends of adjacent nanostructures separated by area 50. In some cases, the nanostructures may be nanotubes joined by DNA linker molecules. Upon exposure to analyte 60, at least a portion of device may interact with the analyte to produce a change in a property of interaction between the nanostructures, producing a conductive network with decreased resistance and increased conductivity (e.g., signal generation). The increase in conductivity may be detected by amperometry (measurement over time of the current intensity, I, between two electrodes at a constant potential, V), or other methods.

In some embodiments, methods for determination of analytes are provided. As used herein, the term “determining” generally refers to the analysis of a species or signal, for example, quantitatively or qualitatively (whether an analyte is present and/or in what amount or concentration), and/or the detection of the presence or absence of the species or signals. “Determining” may also refer to the analysis of an interaction between two or more species or signals, for example, quantitatively or qualitatively, and/or by detecting the presence or absence of the interaction. For example, the method may include the use of a device capable of producing a first, determinable signal (e.g., a reference signal), such as an electrical signal, an optical signal, or the like, in the absence of analyte. The device may then be exposed to an analyte, wherein the analyte may interact with one or more components of the device to cause a change in the signal produced by the device. Determination of the change in the signal may then determine the analyte. The signal may, in some cases, provide information relating to the presence, identity, amount, and/or other characteristic of the analyte.

In some embodiments, the change in signal may occur upon interaction between analyte and at least a portion or component of the device (e.g., linker, probe, etc.). For example, the analyte may contact or may bind to a portion of the device. In some cases, the interaction between the device and the analyte may comprise a reaction, such as a oxidation, reduction, or polymerization reaction. For example, the method may involve exposure of the device to an environment suspected of containing an analyte, wherein the analyte, if present, interacts with the nanostructure network to produce a change in the electrochemical communication (e.g., conductivity) between adjacent nanostructures, which generates a determinable signal. In some cases, the device may be exposed to the analyte by contact with or immersion in a solution comprising the analyte, probe, and/or precursor moiety.

In some cases, the determinable signal comprises a change (e.g., increase, decrease) in an electrochemical property of the network of nanostructures. The electrochemical property may be a change in conductivity, a change in resistance, and/or a change in capacitance. In some cases, the interaction between the analyte and the device may result in an increase in the conductivity of the nanostructure network. In some cases, the interaction may result in a decrease in the conductivity of the nanostructure network. In other embodiments, the interaction may result in an increase in the capacitance of the nanostructure network. In other embodiments, the interaction may result in a decrease in the capacitance of the nanostructure network. In other embodiments, the interaction may result in an increase in the resistance of the nanostructure network. In other embodiments, the interaction may result in a decrease in the resistance of the nanostructure network. In some embodiments, the device undergoes an increase in conductivity, resistance, or capacitance of at least 5%, at least 10%, at least 25%, at least 50%, at least 75%, at least 100%, at least 200%, at least 300%, at least 400%, or, at least 500%, relative to the conductivity, resistance or capacitance of the device in the absence of analyte.

In some embodiments, the determinable signal comprises a change in a luminescent property of the composition.

As described herein, the analyte may interact with a portion of the device to produce a species that affects a property of interaction between the nanostructures. In some cases, the species may affect an electrochemical property of the device. For example, in the absence of an analyte, the device may comprise a network of nanostructures positioned relative to each other so as to form an interrupted circuit. That is, the nanostructures may be arranged with adjacent nanostructures separated by a distance, such that electrochemical communication between adjacent nanostructures is minimized or prevented. In the presence of an analyte, the analyte may interact with at least a portion of the device to produce a species (e.g., a conductive species) that affects the electrochemical communication between the nanostructures. The species may be, for example, a metal, metal ion, metal-containing compound, or a conducting polymer.

In some cases, the species may be formed proximate the area between adjacent nanostructures of the network, in an amount sufficient to affect one or more properties of interaction between adjacent nanostructures. For example, the method may involve directed deposition of a species in the spaces between adjacent nanostructures, in the presence of analyte. That is, the species may be formed proximate the area between adjacent nanostructures, and may not be formed, or may be formed to a lesser extent, at other locations of the device. In some cases, upon interaction with an analyte, a conductive species may be formed near or within the area between adjacent nanostructures to form an uninterrupted circuit along the nanostructure network. For example, the device may be in contact with a probe and precursor moiety, which may interact to generate the species. In the absence of analyte, the species may be generated in low amounts, and/or at locations not sufficiently proximate the nanostructure network, such that the species has minimal or no effect on the interaction between nanostructures. In some cases, the species may diffuse away from the nanostructure network, in the absence of analyte. By contrast, in the presence of analyte, the probe and/or precursor moiety may be brought in close proximity to the area between adjacent nanostructures, such that the species may be produced in a localized manner, i.e., at the junction between nanostructures, and/or may be substantially non-diffusive in solution, thereby affecting interaction between nanostructures and creating a determinable signal.

Such localized formation of the species may occur upon interaction of the analyte with at least a portion of the nanostructure network and the probe. For example, a first portion of the analyte may bind the linker and a second portion of the analyte may bind the probe, bringing the components in close proximity to one another. In some embodiments, the probe is brought in close proximity to the area between adjacent nanostructures, such that species generated by the probe may be deposited or formed within the area between adjacent nanostructures. As described herein, such localized formation of the species may result in a determinable change in the electrochemical properties (e.g., conductivity, capacitance) of the device.

The analyte may interact with the probe and the nanostructure network (e.g., linker) via any chemical or biological interaction, including covalent or non-covalent bonding. In some embodiments, the interaction between the analyte and the probe or nanostructure network may comprise formation of a bond, such as a covalent bond (e.g. carbon-carbon, carbon-oxygen, oxygen-silicon, sulfur-sulfur, phosphorus-nitrogen, carbon-nitrogen, metal-oxygen or other covalent bonds), an ionic bond, a hydrogen bond (e.g., between hydroxyl, amine, carboxyl, thiol and/or similar functional groups, for example), a dative bond (e.g. complexation or chelation between metal ions and monodentate or multidentate ligands), or the like. The interaction may also comprise Van der Waals interactions. In some embodiments, the analyte interacts with the probe and/or nanostructure network via hydrogen bonding. In some embodiments, the analyte interacts with the probe and/or nanostructure network via DNA hybridization.

The probe and/or nanostructure network may also be capable of biologically binding an analyte via an interaction that occurs between pairs of biological molecules including proteins, nucleic acids, glycoproteins, carbohydrates, hormones, and the like. Specific examples of biological binding pairs include an antibody/peptide pair, an antibody/antigen pair, an antibody fragment/antigen pair, an antibody/antigen fragment pair, an antibody fragment/antigen fragment pair, an antibody/hapten pair, an enzyme/substrate pair, an enzyme/inhibitor pair, an enzyme/cofactor pair, a protein/substrate pair, a nucleic acid/nucleic acid pair, a protein/nucleic acid pair, a peptide/peptide pair, a protein/protein pair, a small molecule/protein pair, a glutathione/GST pair, an anti-GFP/GFP fusion protein pair, a Myc/Max pair, a maltose/maltose binding protein pair, a carbohydrate/protein pair, a carbohydrate derivative/protein pair, a metal binding tag/metal/chelate, a peptide tag/metal ion-metal chelate pair, a peptide/NTA pair, a lectin/carbohydrate pair, a receptor/hormone pair, a receptor/effector pair, a complementary nucleic acid/nucleic acid pair, a ligand/cell surface receptor pair, a virus/ligand pair, a Protein A/antibody pair, a Protein G/antibody pair, a Protein L/antibody pair, an Fc receptor/antibody pair, a biotin/avidin pair, a biotin/streptavidin pair, a drug/target pair, a zinc finger/nucleic acid pair, a small molecule/peptide pair, a small molecule/protein pair, a small molecule/target pair, a carbohydrate/protein pair such as maltose/MBP (maltose binding protein), a small molecule/target pair, or a metal ion/chelating agent pair. In an illustrative embodiment, the analyte may comprise an entity, such as biotin that specifically binds to a complementary entity, such as avidin or streptavidin, on the probe or nanostructure network.

In one set of embodiments, the device may include a nanotube-DNA network, as well as an oligonucleotide-functionalized probe. FIG. 1A (path I) shows an illustrative embodiment, wherein device 10 comprises nanotubes joined by single-stranded DNA linkers, as well as Au nanoparticles 70 substituted with single-stranded DNA molecules 72. In the presence of single-stranded DNA analyte 60, the DNA linker hybridizes with a first portion of analyte 60, while a second portion of analyte 60 hybridizes with single-stranded DNA molecule 72. Such interaction positions probe 70 in close proximity to the space between adjacent nanotubes. Au nanoparticles 70 then convert precursor moieties, such as Ag⁺, present within the device, thereby depositing silver metal at the junction between adjacent nanostructures. Upon silver deposition, a network of uninterrupted nanotube wires connecting two electrodes may be formed, producing a determinable change in the conductivity or capacitance of the device. FIG. 1A (path II) shows another embodiment, wherein probe 90 comprises a peroxidase enzyme such as horseradish peroxidase, which is capable of reducing silver ions to silver. Similarly, the DNA linker may hybridize with a first portion of analyte 60, while a second portion of analyte 60 hybridizes with single-stranded DNA molecule 92 on analyte 90. Use of such devices can allow for highly sensitive and selective detection of analytes such as, for example, Ebola virus DNA.

In some embodiments, the device comprises a first electrode and a second electrode in electrochemical communication with the network of nanostructures, such that the determinable signal comprises a change in resistance to current flow between the first and second electrodes. For example, the resistance may decrease upon exposure to an analyte. In some cases, methods described herein may determine an analyte with relatively high selectivity and/or specificity. For example, the device may comprise a network of nanostructures that is responsive to a particular type of analyte and is substantially unresponsive to other analytes or is responsive to a lesser degree, such that the change in signal may be attributed to an interaction between the network of nanostructures and the desired analyte. In some cases, the method may involve determination of more than one type of analyte present within a sample. For example, the interaction between a first analyte and the nanostructure network may give a first change in the properties (e.g., electrical properties) of the nanostructure network, while the interaction between a second analyte and the nanostructure network may give a second, different change in the properties of the nanostructure network, such that distinguishable changes in signal may be determined for both the first and second analytes.

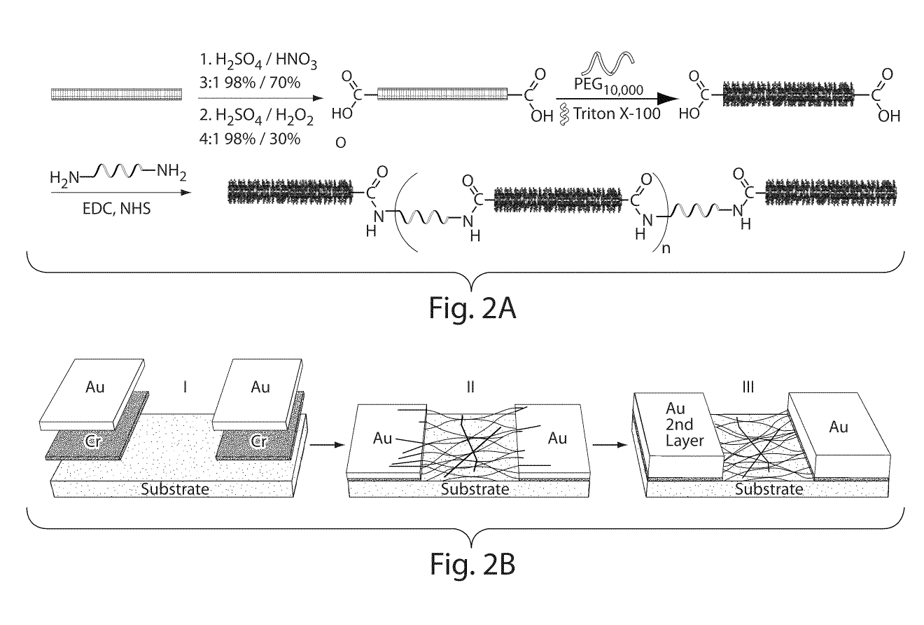

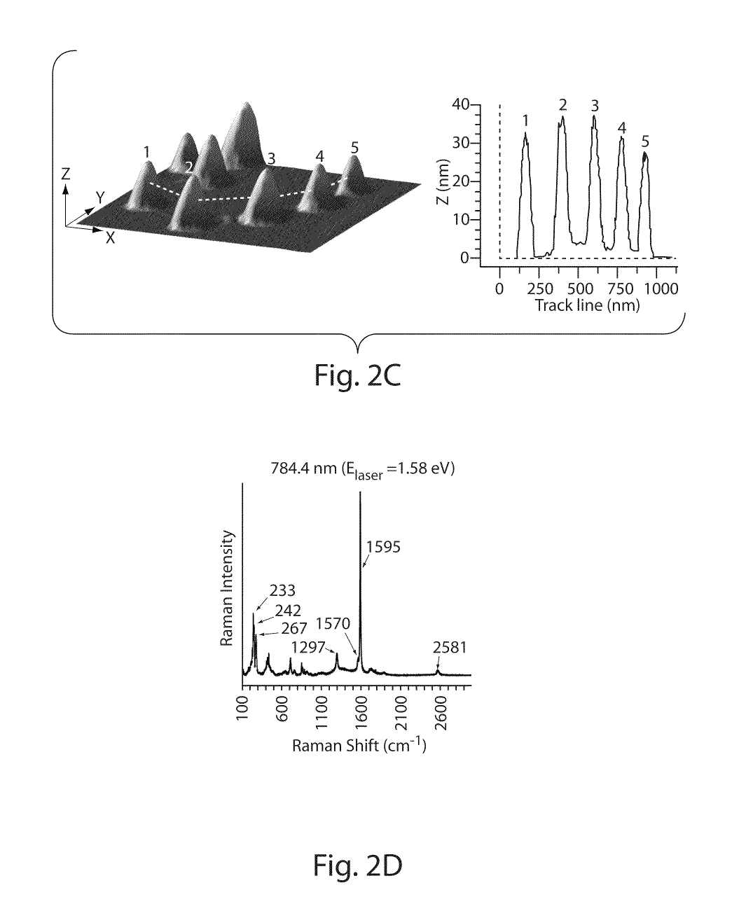

Method for synthesizing a nanostructure network are also provided. In some cases, the method may involve reacting a nanostructure species comprising at least two reactive sites with a linker species comprising at least two reactive sites. In some cases, the at least two reactive sites of the nanostructure species are arranged at or near terminal ends of the nanostructures. The reactive site may comprise an amine, a hydroxyl group, a carbonyl group, an olefin, or the like. In some embodiments, the reactive site may be a biological molecule, including biological molecules capable of forming hydrogen bonds (e.g., single-strand DNA). In some cases, the nanostructures and linkers react to form covalent bonds therebetween. For example, covalent bonds between nanostructures and linkers may be formed via condensation reactions, cycloaddition reactions (e.g., dipolar cycloadditions, “click” chemistry), Wittig reactions, radical reactions, metal-catalyzed coupling reactions, metathesis reactions, free radical addition of thiols to alkenes, biotin-avidin interactions, conjugate addition reactions to electron poor double bonds, Diels Alder reactions, 1,3-dipolar cycloadditions, amide forming reactions, other polymerization reactions, and the like. FIG. 2A shows an illustrative embodiment, wherein a nanotube comprising carboxylic acid groups at or near the terminal ends of the nanotubes is reacted with a single-stranded DNA molecule substituted with amine groups at its terminal ends.

In some cases, the nanostructures and linkers react to form non-covalent bonds therebetween. For example, the nanostructures and linkers may comprise complementary single-strand DNA groups, such that the nanostructures and linkers assemble to form a network via DNA hybridization. In an illustrative embodiment, a set of nanotubes functionalized with a plurality of single-strand DNA groups may be combined with a different set of nanotubes functionalized with complementary single-strand DNA groups, such that the two sets of nanotubes assemble via DNA hybridization to form a network. In another embodiment, nanoparticles (e.g., gold nanoparticles) functionalized with a plurality of single-strand DNA groups may be combined with nanotubes functionalized with complementary single-strand DNA groups.

The types and relative amounts of nanostructure species and linker species may be varied to produce a network having a desired set of properties and to control the number and types of groups present within the network. For example, a nanostructure comprising a binding site for an analyte or a linker species comprising a binding site for an analyte may be added in low amounts, relative to other nanostructure species and/or other linker species, to reduce the number of binding sites present within the network. This may be advantageous in cases where, for example, an analyte-binding event triggers formation of a conductive material between adjacent nanostructures, thereby forming a continuous conductive pathway and/or giving rise to a detectable signal. In some cases, designing a nanostructure network to have a reduced number of analyte binding sites may reduce the number of analyte binding events needed to complete a circuit within the network and/or to produce a detectable signal.

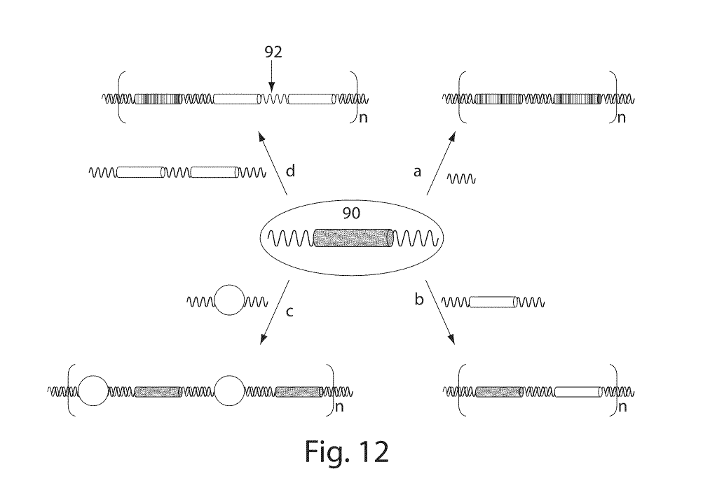

Those of ordinary skill in the art would be able to select the appropriate combination of nanostructure species and linker species, as well as reaction conditions, suitable for use in a particular application. In one set of embodiments, the nanostructures and linkers assemble via DNA hybridization to form the network of nanostructures. In some embodiments, the nanostructures comprise a first set of single-strand DNA groups positioned at or near terminal ends of the nanostructures, and the linkers comprise a second set of single-strand DNA groups, wherein the first set of single-strand DNA groups is complementary to the second set of single-strand DNA groups. FIG. 12 shows various embodiments of the synthesis of nanostructure networks using nanostructure species 90, which includes single-strand DNA groups positioned at or near terminal ends of the nanostructure. Nanostructure species 90 may be exposed to a single-strand DNA molecule that is capable of hybridizing with DNA groups of nanostructure species 90 to form the network. (FIG. 12A) In another embodiment, nanostructure species 90 may be exposed to another nanostructure species comprising single-strand DNA groups that are complementary to those of nanostructure species 90, to form the network. (FIG. 12B) Nanostructure species 90 may also be combined with a nanoparticle substituted with single-strand DNA groups complementary to those of nanostructure species 90 to form the network. (FIG. 12C)

In another embodiment, a linker species may comprise two nanostructures, a single-strand DNA group attached to and positioned between the two nanostructures, and two single-strand DNA groups positioned at the terminal ends of the linker species, where the terminal DNA groups are complementary to the DNA groups of nanostructure species 90, and the interior single-strand DNA group (92) is not complementary to either the terminal DNA groups or the DNA groups of nanostructure species 90. (FIG. 12D) Upon assembly of the nanostructure network via DNA hybridization, single-strand DNA group 92 may be used as a binding site for an analyte. Such a linker species may be incorporated into a network at a lower concentration, relative to nanostructure species and/or other linker species. As described herein, it may be advantageous in some cases to design a network having a relatively low number of analyte binding sites, in order to increase sensitivity of the network for a particular analyte.

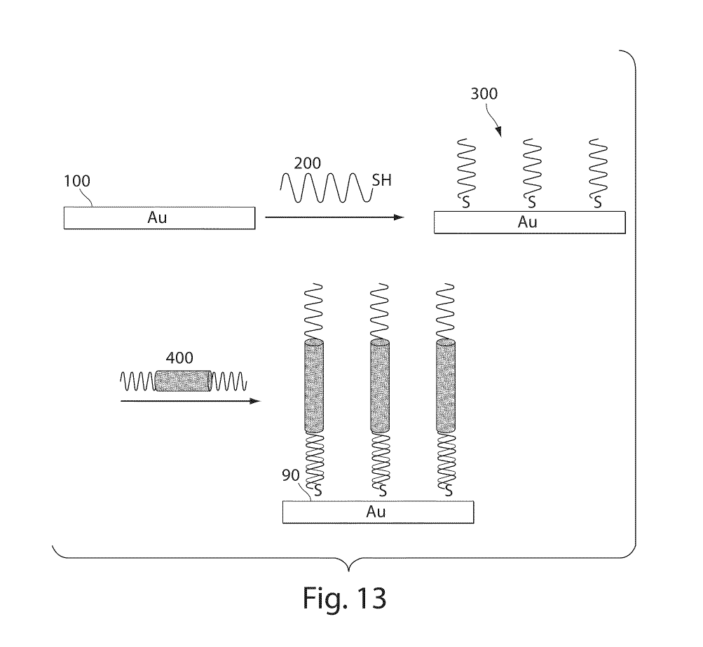

In another set of embodiments, a nanostructure network may be fabricated via directed assembly using a functionalized substrate. As shown by the illustrative embodiment in FIG. 13, gold substrate 100 may be exposed to thiol-substituted single-strand DNA group 200 to produce functionalized substrate 300. Nanostructure species 400 may include single-strand DNA groups that are complementary to those of functionalized substrate 300. Functionalized substrate 300 may then bind nanostructure species 400 via DNA hybridization, and excess nanostructure species may be rinsed away. The substrate may be alternately exposed to various nanostructure species and linker species having complementary single-strand DNA groups in order to produce the nanostructure network. In some cases, the network may be assembled until contact is made with a second substrate or electrode. Those of ordinary skill in the art would be able to select appropriate combinations of substrate materials, nanostructure species, and linker species in order to create nanostructure networks via directed assembly.

The method may further comprise synthesizing and/or processing the nanostructure species, prior to reaction with a linker species. In some embodiments, the method may involve exposing a nanostructure to one or more chemical reactants to install reactive sites at or near the terminal ends of the nanostructures. For example, a nanotube may be treated with a strong acid (e.g., sulfuric acid, nitric acid, mixtures thereof) to install carboxylic-acid groups at the terminal ends of the nanotube. The carboxylic acid groups may be further reacted to install additional groups at the terminal ends, such as biological molecules. In one set of embodiments, the carboxylic acid groups may be reacted with an amine group of a single-strand DNA to form an amide bond, thereby installing single-strand DNA groups at the terminal ends of the nanotube. It should be understood that other types of reactions may be useful in functionalizing nanostructures with reactive sites, biological molecules, analyte binding sites, and the like. For example, the nanostructures may be functionalized via cycloaddition reactions, carbene additions, nitrene additions, DMAD functionalization, halogenation reactions, free radical addition of thiols to alkenes, biotin-avidin interactions, conjugate addition reactions to electron poor double bonds, Diels Alder reactions, 1,3-dipolar cycloadditions, amide forming reactions, and the like

The nanostructure species may be further processed with a protecting material in order to prevent, for example, aggregation of the nanostructures and/or non-specific binding. The protecting material may also enhance compatibility (e.g., solubility, processability) of the nanostructures during fabrication of the device. In some embodiments, a protecting material may be formed on at least a portion of the nanostructure species, including along sidewalls of the nanostructure species. The protecting material may be any hydrophilic material, such as polyethylene glycol, that may be substantially inert to (e.g., unreactive with) the linker species. In some embodiments, the nanostructure may be treated with strong acid to install carboxylic-acid groups at the terminal ends of the nanotube. A protecting material may then be formed on the sidewalls of the nanostructure, such that further reaction of the nanotube may occur primarily at the terminal ends of the nanostructure. Such regioselective functionalization may advantageously allow for the formation of nanostructure networks as described herein, wherein the long axes of the nanostructures are substantially aligned along the long axis of the network.

In some embodiments, the nanostructure network may be a linear polymer comprising nanostructures and linkers. In some embodiments, the nanostructure network may be a branched polymer network. Branches, bends, folds, and other features may be introduced into the network by incorporating species capable of forming secondary structures. For example, biological molecules such as nucleic acids, proteins, and the like, may form loop structures, folded structures, or other secondary structures via intramolecular bonds (e.g., hydrogen bonds). Such biological molecules may be incorporated within the network using the methods described herein. In some cases, DNA groups may be selected to create three-fold, four-fold, or higher, junctions within the network. Those of ordinary skill in the art would be able to select such biological molecules to produce a particular, desired nanostructure network.

Devices of the invention may be fabricated using methods described herein, and/or in combination with other methods known to those of ordinary skill in the art. In some embodiments, methods of the invention may advantageously provide the ability to process materials which may otherwise be insoluble and/or difficult to process. For example, the method may allow for the formation of stable mixtures of nanostructures, such that the nanostructures are readily processible in solution. The mixture may be a solution, a suspension, a dispersion, or the like. In some cases, the method may involve processing a mixture comprising a plurality of nanostructures (e.g., carbon nanotubes) and a linker species to form a nanostructure network as described herein.

The nanostructure network may be processed by various methods, including spin-coating, drop-casting, spray-coating, ink jet printing, electrophoretic deposition, medium scale deposition using a doctor knife, continuous processes and the like. In some cases, the nanostructure network may be formed (e.g., deposited) on a substrate, including an electrode (e.g., interdigitated electrodes), an integrated device, an integrated circuit, or the like. In some embodiments, the nanostructure network may be formed on patterned electrode assemblies. The deposition of metal electrodes may also be performed using known methods, such as evaporation and sputter coating. Array devices for the discrimination of different types of analytes can be created by, for example, ink jet printing of suspensions comprising the nanostructure network and/or any additives (e.g., nanoparticles) on circuitry. For distributed sensory arrays the low power requirements of the devices may be compatible with RFID methods. This versatility may allow for the economical manufacture of devices at any size and in various configurations.

In some embodiments, the nanostructure network may have high mechanical integrity and can be processed as free-standing films.

In some cases, the nanostructure network may be processed to form a thin film on a substrate. A thin film may have a thickness between about 0.1 nm and about 100 um. For example, the thickness of the film may be less than about 100 um, less than about 50 um, less than about 25 um, less than about 10 um, less than about 5 um, less than about 1 um, less than about 500 nm, less than about 250 nm, less than about 100 nm, less than about 50 nm, less than about 25 nm, less than about 10 nm, less than about 5 nm, less than about 2 nm, less than about 1 nm, or in some cases, less than about 0.5 nm. The thickness of the film may or may not be uniform throughout the device. A thin film may be formed using processes such as spin-on methods, chemical vapor deposition, pulsed laser deposition, vacuum plasma spray, wet spray, sputtering, evaporation, or molecular beam epitaxy.

The method may further comprise forming at least one electrode material, for example, on the surface of a substrate or in contact with the nanostructure network. In some cases, at least two electrode materials, or more, are formed on the surface of a substrate or in contact with the nanostructure network. The electrode materials, and other components of the device, may be formed at any time during the fabrication process to produce devices as described herein, or devices having an alternative arrangement. For example, the nanostructure network, electrode materials(s) (e.g., source electrode, drain electrode, gate electrode, etc.), and/or an insulating material may be fabricated in any order to produce a device as described herein. In some embodiments, the electrode material may be formed on a substrate prior to formation of the nanostructure network. In some embodiments, the electrode may be formed on the nanostructure network. In some embodiments, the nanostructure network may be sandwiched between layers of electrode material, i.e., an electrode may be formed on a substrate, followed by formation of the nanostructure network in contact with the electrode, followed by formation of a second electrode layer in contact with the nanostructure network to seal the structure. The electrode material(s) may be deposited onto any component of the device using methods known in the art, such as electroplating or lithography methods known in the art.

As used herein, the term “electrode” or “electrode material” refers to a composition, which, when connected to an electronic device, is able to sense a current or charge and convert it to a signal. An electrode may be comprised of a conductive material or combination of materials such as, for example, metals. Non-limiting examples of suitable metals include gold, copper, silver, platinum, titanium, nickel, cadmium, tin, and the like. The electrodes may also be any other metals and/or non-metals known to those of ordinary skill in the art as conductive (e.g. ceramics). The electrodes may be deposited on a surface via vacuum deposition processes (e.g., sputtering and evaporation) or solution deposition (e.g., electroplating or electroless processes). In a specific example, gold electrodes are deposited by sputter-coating.

In some embodiments, additional components may be formed in order to enhance the stability and/or mechanical integrity of the device. For example, a material capable of enhancing bonding of the nanostructure network or electrode material to each other, or to the surface of the substrate, may be used. As an illustrative embodiment, a chrome later may be deposited on a substrate prior to deposition of an electrode material (e.g., gold) in order to achieve efficient bonding to the substrate and to minimize or prevent erosion of the electrode layer upon exposure to various environments, such as aqueous environments. Those of skill in the art would be able to select additional components suitable for use in the invention.

Systems comprising two or more devices may also be fabricated, wherein each device comprises a first electrode, a second electrode, nanostructure network, precursor moiety, and/or probe, as described herein. In some cases, the individual devices of the system may be substantially identical. For example, the individual devices may be constructed to interact with the same analyte. In some cases, the individual devices of the system may be different, such that each individual device may selectively interact with a particular type of analyte. This may be accomplished, for example, by fabricating a plurality of devices, each comprising a nanostructure network, precursor moiety, and/or probe responsive to a different analyte. For example, each individual device may be capable of interacting with a particular analyte, and may interact with other types of analytes to a different (e.g., lesser) extent. This may be useful in determining two or more different types of analytes present in a single sample.

Devices as described herein may have various device configurations, and may be selected to suit a particular application. For example, the nanostructure network may be fabricated such that a first and the second electrode are in electrochemical communication with the nanostructure network. “Electrochemical communication,” as used herein, refers to materials that are in sufficient communication with each other, such that the transfer of electrons and/or protons and/or other charged moieties can occur between the two materials. For example, the first and second electrodes may not contact one another but may be in electrochemical communication with one another via the nanostructure network, such that upon application of a voltage between the first and second electrode, a current flows from the one electrode through the nanostructure network to the other electrode. In some instances, the first electrode may be a source electrode and the second electrode may be a drain electrode. In some instances, the nanostructure network is placed on a substrate. Non-limiting embodiments of devices are described more fully below.

In some embodiments, simple screening tests may be conducted to select appropriate nanostructure networks (e.g., nanostructures, linkers), probes, precursor moieties, other additives, device configuration, sets of conditions, etc., to suit a particular application. In some cases, a material or device may be screened to determine the sensitivity and/or stability of the material or device. In some cases, a material (and/or device) may be selected based on an ability to detect one or more types of analytes. For example, the ability of a device to detect an analyte may be determined by comparing the signal (e.g., conductivity) of the device prior to and following exposure to the analyte. In another example, a device may be exposed to varying concentrations of the analyte to determine the sensitivity of the device. As another example, a first device and a second device may be provided, wherein the second device comprises a different material (e.g., nanostructure, linker, probe, precursor moiety, additive, electrode material, etc.) and/or configuration (e.g., relative position of components, or additional component such as a gate electrode or insulating material, etc.) as compared to the first device. Signals produced by the first and second devices prior to and following exposure to an analyte (e.g., percent change in signal upon exposure to an analyte, baseline signal, time required for following exposure to the analyte for the signal to return to baseline, etc.) may then be compared to determine differences between the performance of first and second devices.

Devices and compositions described herein may be useful for the detection of analytes (e.g., DNA) at the femtomolar level and can provide a simple and cost effective method for the detection of, for example, infective agents. Sensitivity of the devices to analytes can be further enhanced by regulating the assembly of the nanostructure network by varying the ratio between metallic to semiconducting nanostructures, the length of the nanostructures, the relative concentrations of nanostructures and linkers network, the type of nanostructures used, and the device gap size between the two electrodes. In addition, the area between adjacent nanostructures is flexible and easily amenable to modification for different analytes by varying the length, sequence, structure, and/or identity of the linkers. In networks comprising single-strand DNA groups useful as binding sites for analytes, modifications to the DNA molecular recognition domain (e.g., length, nucleotide sequence, etc.) may readily be made to suit a particular application (e.g., analyte). Additionally, the potential for using different probes, including enzymes such as alkaline phosphatase, glucose oxidase or DNAzymes, which can also deposit gold and silver ions, can provide many new avenues for the construction of hybrid biocatalytic-nanostructured devices. It should be understood that DNA detection is described herein by way of example only, and that the potential for applications beyond DNA detection are vast. Devices comprising networks of nanostructures, as described herein, may be useful in a wide range of applications, including transparent electrode assemblies and highly conductive nanowire networks.

The analyte may be a chemical or biological analyte, or any species capable of interacting with at least a portion of the device to produce a change in a property of interaction between adjacent nanostructures in a network. The term “analyte,” may refer to any chemical, biochemical, or biological entity (e.g. a molecule) to be analyzed. In some cases, the nanostructure network may be selected to have high specificity for the analyte, and may be a chemical or biological sensor, for example. In some embodiments, the analyte is capable of interacting with a portion of the nanostructure network (e.g., linker) and a portion of the probe (e.g., a probe comprising a nucleic acid molecule). The analyte may be a biological molecule, such as a nucleic acid molecule (e.g., DNA or RNA), or an organic molecule (e.g., a small organic molecule).

In some embodiments, the devices and methods described herein may be capable of determining analytes, wherein the analyte concentration is 1000 mM or less, 100 mM or less, 10 mM or less, 1 mM or less, 1000 μM or less, 100 μM or less, 10 μM or less, 1 μM or less, 1000 nM or less, 100 nM or less, 10 nM or less, 1 nM or less, 1000 pM or less, 100 pM or less, 10 pM or less, 1 pM or less, 1000 fM or less, 100 fM or less, 50 fM or less, 25 fM or less, or, in some cases, 15 fM of less. In some cases, the analyte concentration is 10 fM.

As used herein, the term “nanostructure” refers to any chemical structure having at least one dimension on the order of nanometers. In some cases, the nanostructure has an elongated chemical structure having a diameter on the order of nanometers and a length on the order of microns to millimeters, resulting in an aspect ratio greater than 10, 100, 1000, 10,000, or greater. In some cases, the nanostructure may have a diameter less than 1 μm, less than 100 nm, 50 nm, less than 25 nm, less than 10 nm, or, in some cases, less than 1 nm. The nanostructure may have a cylindrical or pseudo-cylindrical shape. In some cases, the nanostructure may be a nanotube, such as a carbon nanotube. In some cases, the nanostructure is a nanorod, nanowire (e.g., metal nanowire), or nanoribbon. In some cases, the nanostructure is a nanoparticle.

As used herein, the term “nanotube” is given its ordinary meaning in the art and refers to a substantially cylindrical molecule, in some cases, comprising a fused network of six-membered aromatic rings. In some cases, the nanotubes may resemble a sheet of graphite rolled up into a seamless cylindrical structure. It should be understood that the nanotube may also comprise rings other than six-membered rings. Typically, at least one end of the nanotube may be capped, i.e., with a curved or nonplanar aromatic group. Nanotubes may have a diameter of the order of nanometers and a length on the order of millimeters, resulting in an aspect ratio greater than about 100, greater than about 1000, greater than about 10,000, or greater. The term “nanotube” includes single-walled nanotubes (SWNTs), multi-walled nanotubes (MWNTs) (e.g., concentric nanotubes), carbon nanotubes, inorganic derivatives thereof, inorganic nanotubes, and the like. The nanotube may be a conductive nanotube. In some cases, the nanotube may be chiral. In some embodiments, the nanotube is a carbon nanotube, such as a single-walled carbon nanotube or a multi-walled carbon nanotube (e.g., a double-walled carbon nanotube).

The nanotubes may be functionalized or substituted with a wide range of functional groups. Examples of functional groups that nanotubes may be substituted with include peptides, proteins, DNA, RNA, peptide nucleic acids (PNA), metal complexes, ligands for metals, ligands for proteins, antibodies, polarizable aromatics, crown ethers, hydroxylamines, polymers, initiators for polymerizations, liquid crystals, fluorocarbons, synthetic receptors, and the like. The properties of the nanotubes may also be tailored based on the substitution of the fused, aromatic network. Those skilled in the art would recognize what types of functional groups would afford a particular, desired property, such as increased solubility, or the ability to determine an analyte. In some embodiments, the substituted carbon nanotube comprises a binding site. The binding site may be positioned at or near a terminal end of the nanotube, or along the sidewalls of the nanotube. In some embodiments, substituted carbon nanotubes may be readily processed in a fluid carrier. That is, dispersions of substituted carbon nanotubes may be formed.

Substituted carbon nanotubes may be synthesized using various methods, including those described in Zhang et al., J. Am. Chem. Soc. 2007, 129(25), 7714; International Publication No. WO2008/133779; International Publication No. WO/2009/136978, the contents of which applications are incorporated herein by reference in their entirety for all purposes. Nanoparticles described herein may have any particle size or average particle size suitable for use in a particular application. As used herein, “particle size” refers to the largest characteristic dimension (e.g., diameter) that can be measured along any orientation of a particle. Particle size may be determined by, for example, dynamic light scattering. For example, the nanoparticle may have a particle size in the range from about 10 to about 1000 nm, about 10 to about 500 nm, about 10 to about 250 nm, about 10 to about 100 nm, or about 10 to about 50 nm. In some cases, the nanoparticle may have a particle size of about 30 nm.

Polymers or polymer materials, as used herein, refer to extended molecular structures comprising a backbone (e.g., non-conjugated backbone, conjugated backbone) which optionally contain pendant side groups, where “backbone” refers to the longest continuous bond pathway of the polymer. In some embodiments, the polymer is substantially non-conjugated or has a non-conjugated backbone. In some embodiments, at least a portion of the polymer is conjugated, i.e. the polymer has at least one portion along which electron density or electronic charge can be conducted, where the electronic charge is referred to as being “delocalized.” Such polymers may also be referred to as “conducting polymers.” A polymer may be “pi-conjugated,” where atoms of the backbone include p-orbitals participating in conjugation and have sufficient overlap with adjacent conjugated p-orbitals. Examples of conducting polymers include polyarylenes, polyarylene vinylenes, and polyarylene ethynylenes. Some specific examples of conducting polymers include polythiophene, polypyrrole, polyacetylene, polyphenylene, polyaniline, and substituted derivatives thereof. In some embodiments, the conducting polymer may be polyaniline. It should be understood that other types of conjugated polymers may be used, such as sigma-conjugated polymers.

The polymer can be a homo-polymer or a co-polymer such as a random co-polymer or a block co-polymer. In one embodiment, the polymer is a block co-polymer. An advantageous feature of block co-polymers is that they may mimic a multi-layer structure, wherein each block may be designed to have different band gap components and, by nature of the chemical structure of a block co-polymer, each band gap component is segregated. As described herein, the band gap and/or selectivity for particular analytes can be achieved by modification or incorporation of different polymer types. The polymer compositions can vary continuously to give a tapered block structure and the polymers can be synthesized by either step growth or chain growth methods.

The number average molecular weight of the polymer may be selected to suit a particular application. As used herein, the term “number average molecular weight (Mₙ)” is given its ordinary meaning in the art and refers to the total weight of the polymer molecules in a sample, divided by the total number of polymer molecules in a sample. Those of ordinary skill in the art will be able to select methods for determining the number average molecular weight of a polymer, for example, gel permeation chromatography (GPC). In some cases, the GPC may be calibrated vs. polystyrene standards. In some cases, the number average molecular weight of the polymer is at least about 10,000, at least about 20,000, at least about 25,000, at least about 35,000, at least about 50,000, at least about 70,000, at least about 75,000, at least about 100,000, at least about 110,000, at least about 125,000, or greater.

In some embodiments, various components of the device are formed on a substrate. The substrate can comprise a wide variety of materials, as will be appreciated by those in the art, including printed circuit board (PCB) materials. Suitable substrates include, but are not limited to, fiberglass, Teflon, ceramics, glass, silicon, mica, plastic (including acrylics, polystyrene and copolymers of styrene and other materials, polypropylene, polyethylene, polybutylene, polycarbonate, polyurethanes, and derivatives thereof, etc.), GETEK (a blend of polypropylene oxide and fiberglass), and the like. The device may also comprise an insulating material. The insulating material may be arranged between the nanostructure network and one or more electrodes (e.g., gate electrode) and/or the substrate. In some cases, the insulating material may reduce the mobile ion damage and minimize drift in gas sensor devices and/or may improve physical adhesion of the nanostructure network to the underlying material or substrate. Examples of suitable insulating materials include, but are not limited to, polysilicate glass, silicon dioxide, silicon nitride, and the like.

As used herein, the term “environment” refers to any medium (e.g., solid, liquid, gas) that can be evaluated in accordance with the invention including, such as air or other vapor samples, soil, water, a biological sample, etc. An “environment suspected of containing” a particular component means a sample with respect to which the content of the component is unknown. For example, a gas environment where one or more forms of an analyte may be present, but not known to have the analyte, defines a sample suspected of containing the analyte.

As used herein, the term “reacting” refers to the forming of a bond between two or more components to produce a stable, isolable compound. For example, a first component and a second component may react to form one reaction product comprising the first component and the second component joined by a covalent bond or a non-covalent bond. That is, the term “reacting” does not refer to the interaction of solvents, catalysts, bases, ligands, or other materials which may serve to promote the occurrence of the reaction with the component(s). A “stable, isolable compound” refers to isolated reaction products and does not refer to unstable intermediates or transition states. As an illustrative embodiment, a first component (e.g., nanostructure species) and a second component (e.g., a linker species) may be reacted to form covalent bonds therebetween to form a network of nanostructures, as described herein. In another embodiment, a first component (e.g., nanostructure species) comprising single-strand DNA groups and a second component (e.g., a linker species) comprising single-strand DNA groups that are complementary to those of the first component may be reacted to undergo DNA hybridization to form a network of nanostructures, as described herein.

The terms “carboxyl group,” “carbonyl group,” and “acyl group” are recognized in the art and can include such moieties as can be represented by the general formula:

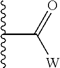

wherein W is H, OH, O-alkyl, O-alkenyl, or a salt thereof. Where W is O-alkyl, the formula represents an “ester.” Where W is OH, the formula represents a “carboxylic acid.” The term “carboxylate” refers to an anionic carboxyl group. In general, where the oxygen atom of the above formula is replaced by sulfur, the formula represents a “thiolcarbonyl” group. Where W is a S-alkyl, the formula represents a “thiolester.” Where W is SH, the formula represents a “thiolcarboxylic acid.” On the other hand, where W is alkyl, the above formula represents a “ketone” group. Where W is hydrogen, the above formula represents an “aldehyde” group.

The terms “amine” and “amino” are art-recognized and refer to both unsubstituted and substituted amines, e.g., a moiety that can be represented by the general formula: N(R′)(R″)(R′″) wherein R′, R″, and R′″ each independently represent a group permitted by the rules of valence.

### EXAMPLES

The following examples describe the synthesis and fabrication of devices for determination of analytes. Within the devices, the sensing surface generally consists of single-stranded DNA (ssDNA) bridging a gap between two single wall carbon nanotubes (SWCNT's) via covalent attachment at their termini leading to the formation of a network of ssDNA linked CNT wires fixed between two gold electrodes. In the presence of the ssDNA analyte, selective binding may occur at the ssDNA junction between contiguous nanotubes resulting in a double-stranded DNA (dsDNA) assembly. The ssDNA analyte has adjacent recognition sequences that are complementary to the nanotube bridging capture strand at one end and to oligonucleotide-functionalized Au nanoparticle or enzyme (horseradish peroxidase) probes at the other end. (FIG. 1B)

Thus, when the device is immersed in a solution consisting of the appropriate probe and analyte, Au nanoparticle probes with a diameter of 30 nm or enzyme probes can hybridize with the analyte recognition domain and occupy the sensing gap between contiguous nanotubes. Silver development allows a means for conductive connection between the interrupted nanotube wires upon analyte detection and provides significant signal amplification. The chemical reduction of Ag (I) promoted by AuNP probes, in the presence of hydroquinone, markedly increase the sensitivity of the device. Silver development has been applied to the sensing of biomolecules, such as nucleic acids and proteins, using colorimetric detection and for the array-based electrical detection of DNA with nanoparticle probes. Similarly, peroxidase enzymes are capable of reducing silver ions to silver metal in the presence of an oxidant leading to the deposition and accumulation of silver metal in a highly localized manner. The use of ssDNA linked CNTs provides flexible control of the recognition gap between contiguous nanotubes allowing for rapid sequence and length variations. In addition, the size of the AuNPs can be modified leading to optimal filling of the analyte sensing junction.

Unless otherwise noted, all the starting materials were obtained from commercial suppliers and used without further purification. Purified HiPco Single-Wall Carbon Nanotubes were purchased from Unidym, Inc. Oligonucleotides were purchased from Integrated DNA Technologies. N-Hydroxysulfosuccinimide sodium salt (sulfo-NHS); 1-(3-dimethylaminopropyl)-3-ethylcarbodiimide hydrochloride (EDC), 98+%; Tween® 20; Triton X-100; polyethylene glycol (PEG) with average mol wt 10,000; sodium dodecylsulfate (SDS); octyltrichlorosilane (OTS), 97%; 6-mercapto-1-hexanol (MCH), 97%; hydrogen peroxide 30 wt % solution in water; sodium nitrate ≧99.0%; sodium phosphate monobasic (anhydrous); sodium phosphate dibasic; magnesium chloride solution (1.00 M±0.01 M); 2,2′-azino-bis(3-ethylbenzthiazoline-6-sulfonic acid) (ABTS); silver enhancer solution A and B; peroxidase from horseradish, Type II (HRP); albumin from bovine serum (BSA) and nonfat-dried bovine milk were purchased from Sigma-Aldrich. Hepes (free acid) was purchased from EMD Biosciences, Inc. Sodium chloride was obtained from Mallinckrodt Baker, Inc. Au-NPs (average diameter of 30 nm) were purchased from Ted Pella, Inc. EnzMet™ kit was acquired from Nanoprobes, Inc. Microcon centrifugal filter devices and polycarbonate membrane filters (0.6 μm) were purchased from Millipore. NAP-5 columns were obtained from GE Healthcare. N-[g-Maleimidobutyryloxy]sulfosuccinimide ester (Sulfo-GMBS) was obtained from Pierce Biotechnology, Inc. Phosphate-buffered saline (PBS×10) without calcium and magnesium was purchased from Mediatech, Inc. Ultrapure water from a NANOpure Diamond (Barnstead) source was used throughout all of the experiments.

Devices were fabricated with ATC ORION 5 Sputtering system (AJA International, Inc.) using chrome and gold as sputtering substrates. All sonication procedures were conducted with an ultrasonic bath (Branson Ultrasonics Corporation, model 3510). Glass substrates and devices were cleaned with UV and Ozone using a UVO Cleaner® model No. 42 (Jellght Company, Inc.). Resistance measurements were recorded with a Fluke 287 True-RMS Electronic Logging Multimeter with TrendCapture (Fluke, Everett, Wash.). UV/Vis spectra were recorded on an Agilent 8453 diode-array spectrophotometer.

Confocal Raman microscopy was performed using a Horiba Jobin Yvon Raman confocal microscope (model LabRAM-HR) with 784.4 nm (1.58 eV), 632.8 nm (1.96 eV), and 532.2 nm (2.33 eV) lasers as the excitation light sources. A ×50 objective was used for imaging with a pin hole size of 300 microns. All atomic force microscopy (AFM) imaging measurements were performed at room temperature using a Multimode scanning probe microscope with a Nanoscope 3A controller (Digital Instruments/Veeco Probes). AFM topographical images were taken on samples deposited on freshly cleaved grade v-4 mica surfaces (Structure Probe, Inc.) followed by spin-coating the solution of interest at 2000 rpm. Images were taken with Ultrasharp SiN AFM tips (MikroMasch) in tapping mode at their resonant frequency, and these images were analyzed with WsXM SPIP software (Nanotec) (please see Horcas et al., Rev. Sci. Instrum. 2007, 78, 013705, the contents of which are incorporated herein by reference in its entirety for all purposes).

The following DNA sequences were utilized:

Probe DNA:
5′-HS-AAAAAAAAAATTGTTGATACTGTTC-3′
Linker DNA (capture strand):
5′-H₂N(CH₂)₆-AAGAATCCAACATTTACTCCAAAAA-NH₂-3′
Target DNA:
5′-GGAGTAAATGTTGGAGAACAGTATCAACAA-3′
Control DNA:
5′-ATGATTAGGTTGCACTCACACTATTACATCTGGCT-3′

### Match and Mismatch Oligonucleotide Sequences:

Match (target):
5′-GGAGTAAATGTTGGAGAACAGTATCAACAA-3′
1AA mismatch:
5′-GGAGAAAATGTTGGAGAACAGTATCAACAA-3′
1CC mismatch:
5′-GGACTAAATGTTGGAGAACAGTATCAACAA-3′
2AA mismatch:
5′-GGAGAAAAAGTTGGAGAACAGTATCAACAA-3′
3AA mismatch:
5′-GGAGAAAAAGATGGAGAACAGTATCAACAA-3′
Non-match (control):
5′-ATGATTAGGTTGCACTCACACTATTACATCTGGCT-3′

### Buffers

Disulfide cleavage buffer: Phosphate buffer, 170 mM, pH 8.0.
Blocking buffer: 0.1 mM 6-mercapto-1-hexanol, 0.1% Tween 20 (v/v) aqueous solution.
Storage buffer: PBS, 1% BSA (wt/v), 1% Triton X-100 (v/v), pH 7.4.
Assay buffer 1 (Au-NP-DNA probes): PBS, 0.1% Tween 20 (v/v), pH 7.4.
Assay buffer 2 (HRP-DNA probes): PBS, 0.1% nonfat milk, 0.025% Tween 20 (v/v), pH 7.4.
Slide washing buffer A: 0.5 M NaNO₃, 0.01% SDS (wt/v), 0.1% Tween 20 (v/v) in NANOpure water.
Slide washing buffer B: 0.5 M NaNO₃ in NANOpure water.
Slide washing buffer C, 0.1 M NaNO₃ (store at 4° C.) in NANOpure water.

### Example 1

The following example describes the synthesis of a DNA-linked carbon nanotube wire. First, single-walled carbon nanotubes (SWCNTs) were cut and oxidized. Commercial HiPco SWCNTs were cut and etched according to a reported procedure (please see Liu et al., Science 1998, 280, 1253, which is incorporated herein by reference in its entirety for all purposes) to give shortened SWCNTs bearing carboxylic groups at their terminal ends (“SWCNT-COOH”) and also at defect sites on the sidewalls. Specifically, 50 mg of pristine pure HiPco SWCNTs were placed in a solution of 98% H₂SO₄/70% HNO₃ (3:1, 24 mL) at 40° C. and sonicated at 42 kHz for 35 min. The solution was filtered using a 0.6 μm polycarbonate membrane filter and then etched for 30 minutes with a solution of 98% H₂SO₄/30% H₂O₂ (4:1, 20 mL) to remove all carbon particles produced by the first reaction. The resulting diluted nanotube-acid mixture was then filtered using a 0.6 μm polycarbonate membrane filter leaving a SWCNT filter cake. The nanotubes were then rinsed with NANOpure water. Final rinsing was done using ethanol and the resulting filter cake dried in a vacuum desiccator.

Next, the sidewalls of the SWCNT-COOH were shielded using polyethylene glycol. Typically, 0.1% SWCNTs-COOH (wt/v) were placed in an aqueous solution containing 0.25% Triton X-100 (v/v) and 0.25% PEG (10,000 Mᵣ) (wt/v) in a final volume of 1 mL and sonicated for 4 hrs at 42 kHz in an ice bath, followed by centrifugation at 14,000 rpm for 1 h. The supernatant was then collected for the next step, removing a small residual amount of aggregated SWCNTs.

Linker DNA strands were then tethered to terminal ends of the shielded SWCNT-COOH. Generally, 0.05% SWCNTs-COOH (wt/v) were placed in a 0.1 M Hepes buffer solution, pH 7.4 consisting of 0.125% Triton X-100 (v/v), 0.125% PEG (10,000 Mᵣ) (wt/v), 2 pmol of linker DNA, 2 mM EDC, and 5 mM sulfo-NHS in a final volume of 1 mL, and stirred gently overnight in the dark at room temperature. The reaction mix was then purified and separated from the excess coupling reagents using a Microcon centrifugal filter device unit (cut-off MW 100,000), and concentrated to a final volume of 200 μL.

### Example 2

The following example describes Au-nanoparticle functionalization with DNA. Au-nanoparticles (average diameter of 30 nm) were used at a concentration of ˜330 pM (˜2×10⁻¹¹ particles mL⁻¹). The disulfide bonds in all oligonucleotides (Probe DNAs) were reduced prior to mixing with the Au colloid by soaking in 0.1 M DTT with disulfide cleavage buffer for 2-3 h (5 nmol of lyophilized DNA was reduced with 100 μL of fresh 0.1 M DTT). The solutions of deprotected DNA were purified through desalting NAP-5 columns, and the amount of DNA from each column was determined by reading the absorbance of the solutions at 260 nm. A solution of the freshly deprotected DNA was then added to the Au colloid, 1 mL (final concentration of oligonucleotides 3-4 μM) to functionalize the Au nanoparticles, and the mixture was shaken gently overnight at room temperature. Phosphate buffer was added to the nanoparticle solution to obtain a final phosphate concentration of 9 mM, and a surfactant solution was added to obtain a final sodium dodecylsulfate (SDS) concentration of ˜0.1% (wt/v). Finally, the NaCl concentration was brought to 0.2 M in a stepwise manner over 1 day. After the salting process was completed, the Au probes were stored at 4° C.

Prior to the conductance experiment, the Au-nanoparticle probes were purified from excess DNA by centrifugation (four times at 10,000 rpm at 10° C. for 10 min) and washed each time with assay buffer PBS, 0.1% Tween 20, pH 7.4. All Au-NP probes were finally redispersed in assay buffer at a concentration of 1 nM. The exact Au-NP concentration was determined by reading the absorbance of the colloid solution at 530 nm. The molar extinction coefficient for 30 nm particles at 530 nm is 3×10⁹ M⁻¹ cm⁻¹.

### Example 3

In the following example, horseradish peroxidase (HRP) was functionalized with DNA.

The crystal structure of HRP (PDB 1hch) was analyzed, rendered, and solvent accessible surface area calculations were performed using the UCSF Chimera software package (3). E. F. Pettersen, T. D. Goddard, C. C. Huang, G. S. Couch, et al., J Comput Chem 25, 1605-12 (2004). 1.75 nmol of HRP was reacted with 8.75 nmol of Sulfo-GMBS and 17.5 nmol thiolated DNA (Probe DNAs) in a ratio of 1:5:10, for 3 h at room temperature. The excess Sulfo-GMBS and DNA were removed using a Microcon centrifugal filter device unit (cut-off MW 30,000). Prior to modification, the disulfide bonds in all oligonucleotides were reduced by soaking in 0.1 M DTT in disulfide cleavage buffer for 2-3 h (100D of lyophilized DNA is typically reduced with 150 μL of freshly prepared solution of 0.1 M DTT). The deprotected DNA solutions were purified through desalting NAP-5 columns, and the amount of DNA from each column was determined by reading the absorbance of the solutions at 260 nm. The modified HRP was diluted to a final concentration of 4 μM with storage buffer and stored at −30° C.

The number of DNA oligomers per HRP molecule was then determined. Concentrations of 5′-HRP-labeled oligonucleotides (probe DNA) were calculated either by the peak area at 260 nm (DNA concentration) and/or by incubating serial dilutions of the 5′-HRP oligonucleotides. Standard dilutions of native HRP were used for calibration at 402 nm DNA-labeled HRP concentrations were interpolated from the calibration curve. Comparing the spectra of native HRP, free oligonucleotide, and oligonucleotide-modified HRP, the number of DNA strands conjugated per each HRP molecule was determined.

The enzymatic activity of the DNA-HRP conjugates was then compared to native HRP. The activity of the enzyme was determined colorimetrically using a UV-Vis spectrophotometer. A mixture of 8.7 mM ABTS, 0.01% hydrogen peroxide (wt/wt), 0.004% BSA (wt/v), 0.008% Triton X-100 (v/v) and 0.1 nM of the native HRP or the modified HRP, placed in PBS buffer, pH 7.4. The spectrophotometer was adjusted to 414 nm, and increases in absorbance were recorded for 5 min. The concentration of HRP was diluted so that a reasonable reaction rate (usually ΔA/min=0.02-0.04) could be obtained.

### Example 4

The following example described the synthesis of DNA-conjugated nanotubes. FIG. 2A shows a schematic representation summarizing the overall synthesis, including oxidation of nanotubes, shielding of nanotube sidewalls with a protecting material, and nanotube-DNA conjugation. First, HiPco SWCNTs were acid-treated in a mixture of concentrated sulfuric and nitric acid (3:1, 98% and 70%, respectively), subjected to sonication for 35 minutes at 40° C., and etched with a mixture of concentrated sulfuric acid and hydrogen peroxide (4:1 98% and 30%, respectively, 30 min) to give carboxylic acid functionalized nanotube ends and to remove all carbon particles produced by the first reaction. Previous studies have demonstrated that the chemical shortening of SWCNTs by mixtures of strong acids can lead to oxidation at the nanotube ends, providing functionality for the covalent attachment of DNA to the termini. Treatment with strong acids have also been shown to lead to formation of sidewall defect sites.

Next, the oxidized nanotubes were treated with a protecting material to shield the surface of the nanotubes from undesired chemical reactions and non-specific surface adsorption, while also increasing the solubility and processability of the nanotubes. The oxidized nanotubes were incubated with Triton X-100/PEG (Mᵣ=10,000) in an aqueous solution and sonicated for 4 h in an ice bath, shielding the nanotube sidewalls.

Lastly, the oxidized termini of the shielded SWCNTs were coupled to the DNA capture strands bearing a nucleophilic primary amine at the 5′ and 3′ ends. Efficient coupling was achieved using 1-(3-Dimethylaminopropyl)-3-ethylcarbodiimide hydrochloride (EDC) and N-hydroxysulfosuccinimide (sulfo-NHS) resulting in SWCNTs linked by DNA at their termini in a nanowire configuration. The relative cross-sectional area of duplex DNA (−3 nm²) as compared to the diameter of HiPco SWCNTs (between ˜0.7-1.2 nm) provided a basis for monofunctionalization of the nanotube ends.

### Example 5

The following example describes the fabrication of a device for determining a biological analyte. Specifically, a carbon nanotube-DNA network device including oligonucleotide-functionalized Au nanoparticles (AuNPs) and enzymes was fabricated. In the sensor design shown in FIG. 1A, a DNA linked-CNT wire motif forms a network of interrupted carbon nanotube wires connecting two electrodes. Sensing can occur at the DNA junctions linking CNTs, such that only the detection of analyte can allow for silver deposition leading to a conductimetric response.

As shown in FIG. 2B, such devices were fabricated by deposition of (I) electrodes, (II) the nanowire network assembly, and (III) another electrode layer. Devices were fabricated by shadow masking gold electrodes (50 nm Au on 10 nm Cr) with 250 μm gaps on a glass or silicon oxide substrate, which was blocked by octyltrichlorosilane treatment (0.2% OTS) to reduce non-specific binding to the device surface (e.g., to block the surface Si—OH bonds). OTS treatment involved cleaning the chips with hot ethanol, drying with nitrogen, and cleaning for 30 seconds using a UV-Ozone cleaner. Next, the chips were soaked in a solution containing 0.2% OTS (v/v) in toluene for 15 minutes. The chips were washed with pure toluene to remove excess OTS, and dried using nitrogen followed by 30 minutes at 90° C. in an oven. Finally, the chips were soaked in toluene for 5 minutes with sonication using an ultrasonic bath.

The DNA-SWCNT nanowires (0.5 μL, DNA concentration ˜1×10⁻⁸ M) were then spin coated at 2,000 rpm between the device electrodes to form a network and a second layer of gold (75 nm) was deposited on top of the first electrodes effectively fixing the DNA-CNT network in a sandwich-like configuration. (FIG. 2B) Chips were immersed in blocking buffer for 1 h to block the vacant gold surface with MCH and to block the vacant SWCNT surface with Tween 20. Finally, the chips were washed with NANOpure water to remove excess MCH and Tween 20, followed by drying with nitrogen.

In some cases, it was useful to deposit a chrome layer beneath the first gold layer of the electrodes for efficient bonding to the glass substrate and to prevent erosion of the gold layer upon contact with aqueous buffers. Good results were also obtained by treating the devices with an aqueous solution of 0.1 mM mercaptohexanol (MCH) and 0.1% Tween 20 prior to use. The MCH was used to prevent nonspecific silver deposition on the gold electrodes, which can lead to false-positive results, and Tween 20 was used to prevent non-specific binding to the CNT surface.

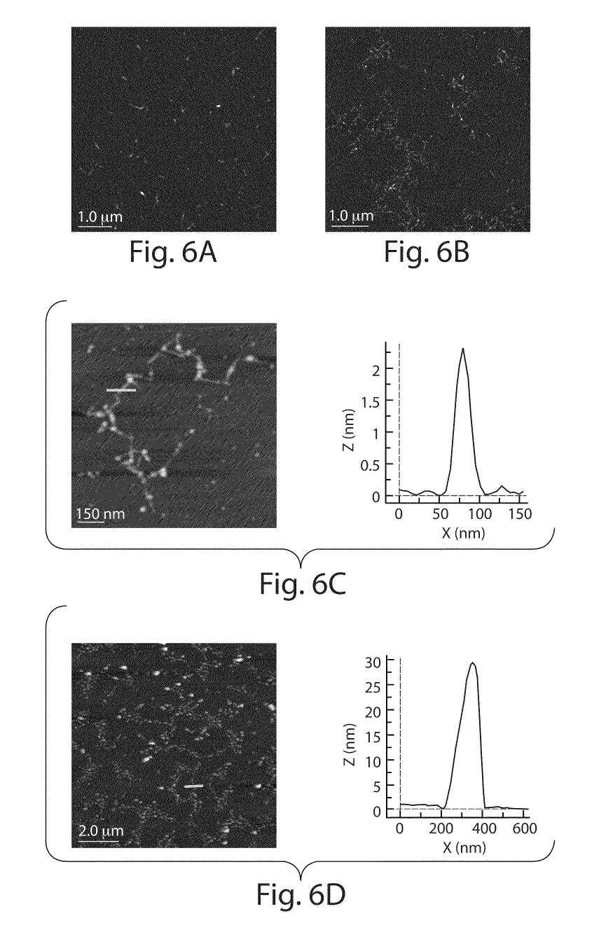

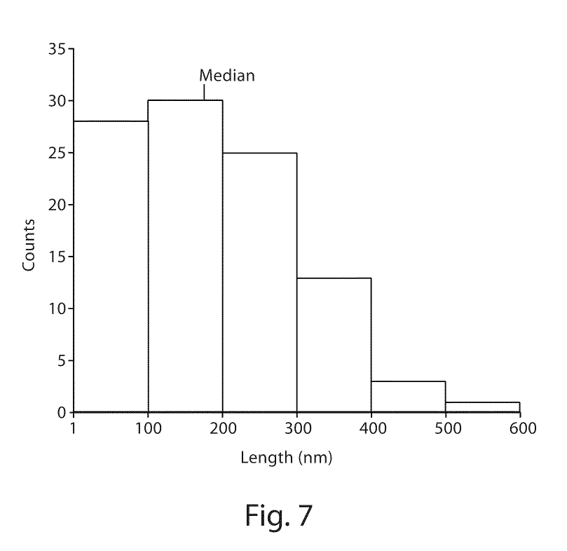

Additional characterization of the DNA-CNT nanowires was performed using AFM and confocal Raman spectroscopy before and after target and probe hybridization. To confirm the nature of the nanowire DNA junctions, target DNA and AuNP probes were utilized. AuNP probes were chosen due to their large diameter (−30 nm) compared to shielded-CNTs (1-5 nm) providing an unambiguous probe for identification of the DNA junctions. FIG. 2C shows DNA-CNT nanowire characterization by AFM utilizing target DNA and AuNP-probe hybridization for DNA junction visualization, including the plot of profile heights along the nanowire-probe assembly. Sequential AuNPs are designated as 1-5 and the trackline is shown in white on the AFM image. FIGS. 6B-C show AFM images of DNA-conjugated CNT nanowires with terminal connections mediated by DNA junctions, as well as height profiles of a single-shielded CNT within the nanowire (as indicated on the AFM image in FIG. 6C). FIG. 6D shows DNA-conjugated CNTs with bound Au nanoparticle probes and target DNA (1 nM), as well as the height profile of a single Au nanoparticle probe bound to a DNA nanowire junction (as indicated on the AFM image in FIG. 6D). The AFM images showed Au nanoparticles bound at each DNA junction, confirming the presence of DNA-CNT nanowires, as well as the presence of contiguous nanotubes with terminal connections mediated by DNA junctions and individual nanotube lengths of ˜200 nm. By contrast, oxidized shielded CNTs were dispersed and separated from adjacent nanotubes after sonication as evidenced by AFM, with average lengths of ˜200 nm (FIG. 6A and FIG. 7).

Additionally, confocal Raman spectroscopy was performed on the mica deposited AFM samples and confirmed the presence of carbon nanotubes. (FIG. 2D)

### Example 5

In the following example, a general DNA detection assay is described. The target DNA in assay buffer (dependant what probe used) was reacted with the device surface followed by incubation at 25° C. for 2 h to allow hybridization between the captured DNA strands and the target DNA. Next, the devices were washed with the same assay buffer (5×1 mL). The corresponding probe was added to the capture DNA-target complexes, and hybridization was allowed to proceed at 25° C. for 1 hr. The devices were washed with assay buffer (5×1 mL), washing buffer A (5×1 mL), washing buffer B (5×1 mL) and washing buffer C (5×1 mL) to remove nonspecifically bound NPs or enzymes and chlorine ions that can react with the silver ions in the next step. Finally, the devices were soaked briefly in NANOpure water and dried with nitrogen.

The resistances were recorded with a Fluke 287 multimeter before and after silver deposition. For probes using NPs, silver deposition was carried out by incubation with a cold enhancer solution of reagents A and B in a 1:1 ratio for 2 minutes. For probes using HRP, silver deposition was accomplished using an EnzMet™ solution of reagents A, B and C in a 1:1:1 ratio for 2 minutes of incubation at room temperature.

### Example 6

As described herein, DNA detection methods are provided which capitalize on metalizing the sensing junctions between contiguous nanotubes using deposition of a conductive species (e.g., metal, polymer) resulting in a highly conductive network. In this example, a DNA detection method utilizing Au nanoparticle probes is described, wherein silver metal is deposited at the sensing junctions between nanotubes. The metalizing process relies on a catalyst probe, i.e., an inorganic catalyst such as Au nanoparticles. a biological catalyst such as peroxidase enzymes.

The DNA sensing scheme utilizing oligonucleotide-labeled AuNP probes as a catalyst reporter is shown in FIG. 1A, path I. As a result of recognition between the analyte and the capture DNA strands on the sensing surface, the catalyst reporter probe hybridized and initiated silver deposition, closing the nanotube gap, leading to a change in conductance. The junction between contiguous nanotubes, dictated by the length of the intervening capture ssDNA, was approximately ˜10 nm. Au nanoparticle sensing probes (30 nm in diameter) allowed for connection of contiguous nanotubes, providing a conductive bridge.

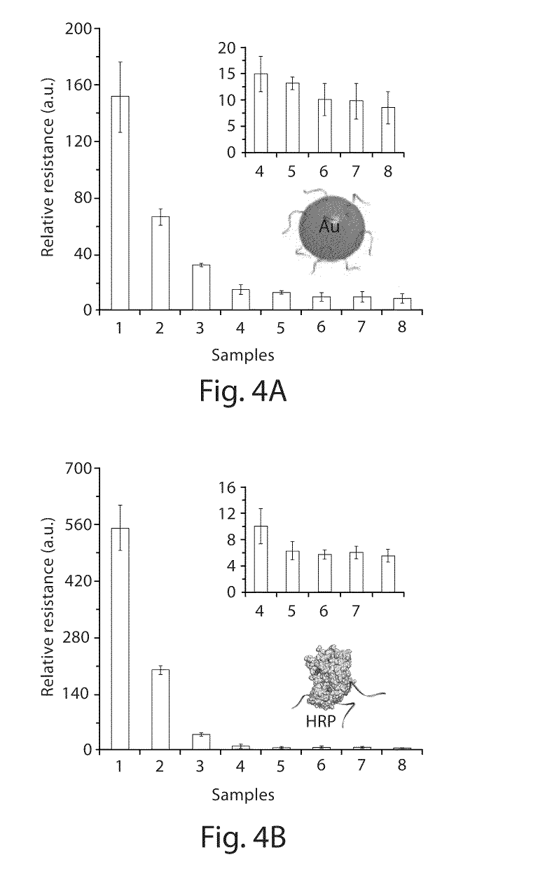

DNA detection studies with Au nanoparticle probes were conducted using a range of DNA analyte concentrations. Conductance changes of the devices after silver deposition were measured using decreasing concentrations of the analyte at a fixed development time of 2 min and are shown in FIG. 4. FIG. 4A shows the conductimetric response data from concentration dependent DNA-detection studies using Au nanoparticle probes. The DNA concentrations for samples 1-5 and control samples 6-8 are summarized in Table 1. All experiments were conducted with a silver development time of 2 min. Based on the relative resistance data shown in FIG. 4A, no significant difference was found between the conductivity in samples 4-8 in accordance with the t test at the 95% confidence level.

That is, introduction of Au nanoparticle probes, which selectively bind in the presence of the analyte, lead to small changes in conductivity. Without wishing to be bound by theory, it is possible that the surfactant layer on the nanotubes and the DNA coating on the Au nanoparticles may have provided an insulating steric barrier between the nanotubes and Au nanoparticles, preventing charge transfer.

TABLE 1
DNA Concentration for Samples 1-8,
using Au nanoparticle probes.

| Sample | DNA Concentration | |
|---|---|---|
| 1 | 100 | pM |
| 2 | 10 | pM |
| 3 | 1 | pM |
| 4 | 10 | fM |
| 5 | 1 | fM |
| 6 (control DNA) | 100 | pM |
| 7 (no DNA) | 100 | pM |
| 8 (no DNA/no probe) | 100 | pM |

### Example 6

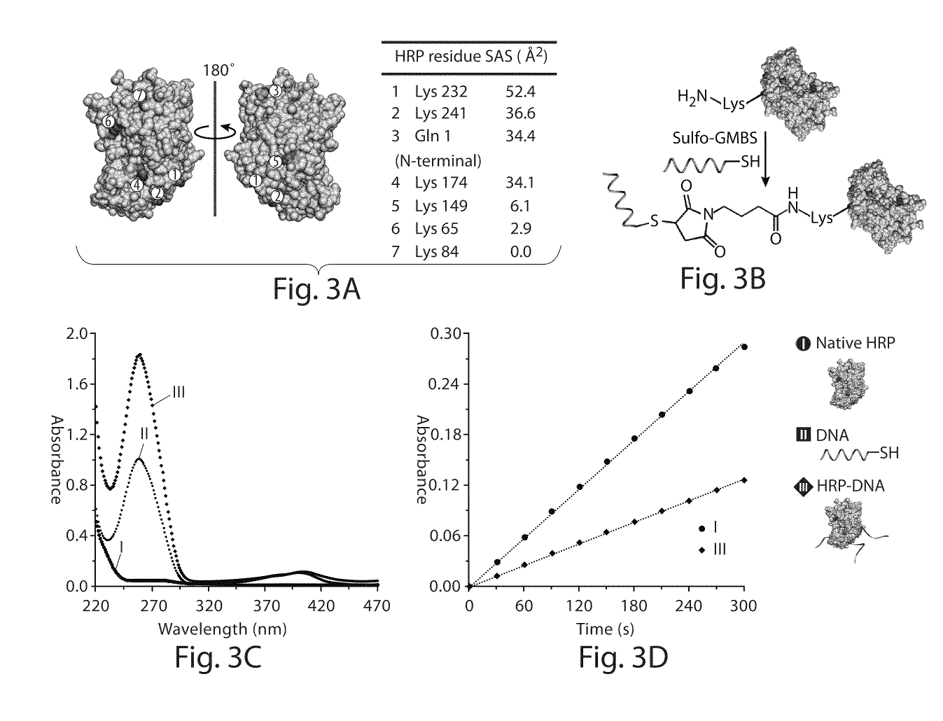

In this example, an oligonucleotide-labeled HRP enzyme probe was synthesized and studied. HRP can act as a biocatalytic reporter to deposit non-diffusible silver metal particles in a highly localized manner at the analyte sensing domain connecting the junction between adjacent nanotubes. FIG. 3A shows the HRP crystal structure (PDB 1hch) with potential nucleophilic residues for DNA conjugation mapped on the surface, and corresponding table of calculated solvent accessible surface areas (SAS) for each residue. The front and back of HRP structure is shown with a 180° rotation. Analysis of the HRP crystal structure reveals three accessible lysine residues out of six on the outer surface of the enzyme and one glutamine at the N-terminus of the protein, allowing the possibility of conjugating up to four oligonucleotide probe strands per enzyme molecule.

FIG. 3A depicts the conjugation strategy used for HRP labeling with DNA. The HRP enzyme was modified with thiolated DNA probes using the heterobifunctional cross-linker N-[γ-maleimidobutyryloxy]sulfosuccinimide ester (sulfo-GMBS). Spectroscopic analysis, using the heme absorbance at λ=403 nm as a reference, allowed for the determination of protein concentration and the absorbance at λ=260 nm for DNA concentration. FIG. 3C shows the UV-Vis determination of DNA stoichiometry for (I) native HRP, (II) DNA, and (III) HRP-DNA. The average stoichiometry of HRP to DNA was found to be ca. 3 to 4 (Avg.=3.7) DNA probe strands per enzyme.

The enzyme activity of HRP-DNA was investigated. Studies to detect ebola virus DNA, by means of simple conductivity measurements, were performed. FIG. 3D shows the enzyme activity assay for (I) native HRP and (III) HRP after DNA conjugation, where HRP activity was reduced to ˜44% of the native enzyme after DNA conjugation.

### Example 7

In this example, DNA detection utilizing a biocatalyzed silver reduction method was applied, employing a DNA probe conjugated to a peroxidase enzyme for analyte detection (FIG. 1A, path II). DNA detection studies with HRP probes were conducted using a range of DNA analyte concentrations. Conductance changes of the devices after silver deposition were measured using decreasing concentrations of the analyte at a fixed development time of 2 min and are shown in FIG. 4.

DNA detection studies with HRP probes were conducted using a range of DNA analyte concentrations. Conductance changes of the devices after silver deposition were measured using decreasing concentrations of the analyte at a fixed development time of 2 min and are shown in FIG. 4. FIG. 4B shows the conductimetric response data from concentration dependent DNA-detection studies using HRP probes. The DNA concentrations for samples 1-5 and control samples 6-8 are summarized in Table 2. All experiments were conducted with a silver development time of 2 min. Based on the relative resistance data shown in FIG. 4B, no significant difference was found between the conductivity in samples 4-8 in accordance with the t test at the 95% confidence level. The error bars represent mean values±s.d.

TABLE 2
DNA Concentration for Samples 1-8, using HRP probes.

| Sample | DNA Concentration | |
|---|---|---|
| 1 | 100 | pM |
| 2 | 10 | pM |
| 3 | 1 | pM |
| 4 | 10 | fM |
| 5 | 1 | fM |
| 6 (control DNA) | 100 | pM |
| 7 (no DNA) | 100 | pM |
| 8 (no DNA/no probe) | 100 | pM |

### Example 8

This example summarizes the results of the DNA assays performed in Examples 6-7. The control experiment where foreign DNA was used as a negative control did not lead to any significant silver deposition and resulted in a low conductivity change. (FIG. 4). In addition, control experiments in the absence of analyte or DNA probe led to negligible conductivity increases. Relative resistance of the device was shown to decrease as a function of decreasing analyte concentration resulting in decreased silver ion reduction and leading to a less conductive nanotube network.

Control experiments revealed that detection of the analyte is specific and can be detected with a limit of 10 fM. Slightly better sensitivity was obtained by utilizing a more specific enzymatic silver deposition probe as opposed to the AuNP probe. FIG. 4A shows a relative resistance value of 152 for detection of nanomolar target DNA (FIG. 4, sample 1) concentrations whereas the enzymatic method showed a relative resistance value of 550. In addition, the detection of DNA at the 10 fm level was slightly better in the case of the HRP probe. The increase in overall relative resistance values for the HRP detection method translated into smaller error bars between replicate measurements and increased sensitivity when compared to the AuNP probe.

### Example 9

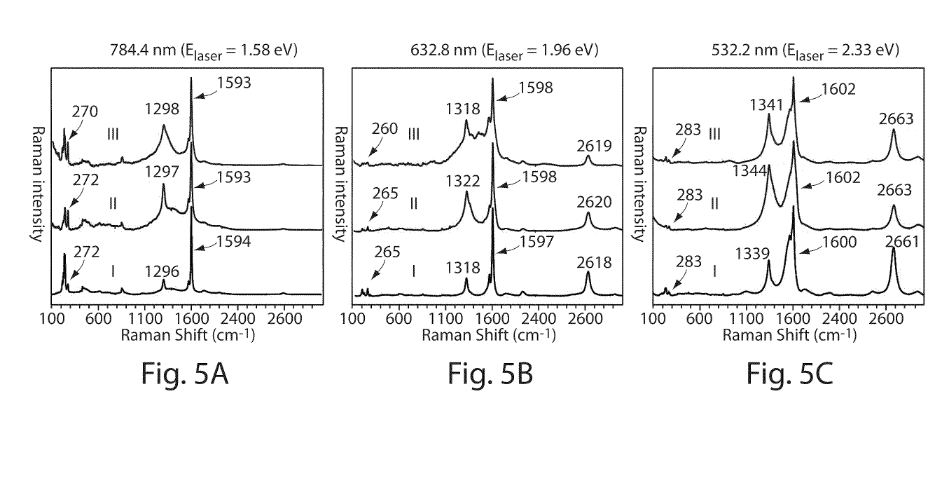

In order to further characterize the sensing gap of the device before and after silver deposition, laser scanning Raman confocal microscopy was performed. Previous resonance Raman studies have demonstrated that SWCNTs filled with metallic silver lead to Fermi level alteration where silver behaves as an electron donor, which is consistent with an increase in electron carrier density. Utilizing laser scanning confocal Raman microscopy, the device junction was characterized before and after analyte (1 nM) detection using AuNP and HRP catalyzed silver deposition. Laser excitation wavelengths of 784.4 nm (1.58 eV), 632.8 nm (1.96 eV), and 532.2 nm (2.33 eV) were used and the result from different resonant conditions are shown in FIG. 5A-C respectively.

FIG. 5A shows the confocal Raman data for device junctions (I) before silver deposition and after silver deposition, using (II) HRP and (III) AuNP silver development probes, and data were obtained using laser wavelength of (a) 784.4 nm (Elaser=1.58 eV), (b) 632.8 nm (Elaser=1.96 eV), and (c) 532.2 nm (Elaser=2.33 eV). An analyte concentration of 1 nM and a ×50 microscope objective was used in all Raman experiments.

Silver modification of the nanotubes after analyte detection resulted in significant changes in the Raman spectra, as shown in FIG. 5. Changes in the RBM, D-band, G-band, and G′-band regions were observed depending on the energy of laser excitation. Using a laser wavelength of 784.4 nm (Eₗₐₛₑᵣ=1.58 eV), the RBM region showed 3 major peaks at 221, 230, and 238 cm⁻¹, which decreased in intensity after silver deposition with Au nanoparticles and HRP. (FIG. 5A) Additionally, an RBM peak at ˜270 nm showed a significant increase in intensity upon silver deposition consistent with increased tube-tube interactions as previously assigned. The intensity of the D-band increased with significant broadening upon silver deposition and shows a slight ˜1-2 cm⁻¹ shift to lower frequency from 1296 cm⁻¹ consistent with previous studies of SWCNTs on silver surfaces. In addition, there was an increase in the D/G ratio upon silver deposition reflecting modification of the nanotube surface. The G+ (1593 cm⁻¹) and G− (˜1564 cm⁻¹) bands also showed a slight decrease in frequency and the ratio of the G−/G+ band intensity increased from 0.15 for the device without silver deposition to 0.31 (HRP) and 0.36 (Au nanoparticle) with silver deposition, which was consistent with an increase in the metallic state of the nanotubes.

Using a laser wavelength of 632.8 nm (Eₗₐₛₑᵣ=1.96 eV) resulted in a significant decrease in the global intensity of the RBM region along with D-band and G-band trends similar to that observed with the 784.4 nm laser. Additionally, there was a large peak in the G′ region using the 632.8 nm laser, which showed a significant decrease in intensity upon silver deposition along with an ˜2 cm⁻¹ shift to higher wavenumbers. Switching to a laser wavelength of 532.2 nm (Eₗₐₛₑᵣ=2.33 eV) resulted in spectral features similar to the 632.8 nm laser.

### Example 10

The following example describes a conductivity-based DNA detection method utilizing carbon nanotube-DNA network devices using oligonucleotide-functionalized enzymes for highly sensitive and selective detection of oligonucleotides analytes. (FIG. 1A(ii)) The sensor design involves a DNA linked-CNT nanowire material, which can be synthesized using a regioselective nanotube functionalization methodology, as described herein. In one case, the sensing surface consists of single-stranded DNA (ssDNA) bridging a gap between two single wall carbon nanotubes (SWCNT's) via covalent attachment at their termini, leading to the formation of a network of ssDNA linked CNT wires fixed between two gold electrodes.

In the presence of the ssDNA analyte, selective binding can occur at the ssDNA junction between contiguous nanotubes resulting in a double-stranded DNA (dsDNA) assembly. The ssDNA analyte has adjacent recognition sequences that are complementary to the nanotube bridging capture strand at one end and to an oligonucleotide-functionalized enzyme (horseradish peroxidase, HRP) probe at the other end (FIG. 3B). Thus, when the device was immersed in a solution consisting of an enzyme probe and analyte, the enzyme probe hybridized with the analyte recognition domain and occupied the sensing gap between contiguous nanotubes. Exposure of the device to the appropriate reagents initiated enzymatic metallization, resulting in conductive connection between the interrupted nanotube wires upon analyte detection and providing significant signal amplification. The overall device assembly process and sensing strategy is outlined in FIGS. 1A and 2B.

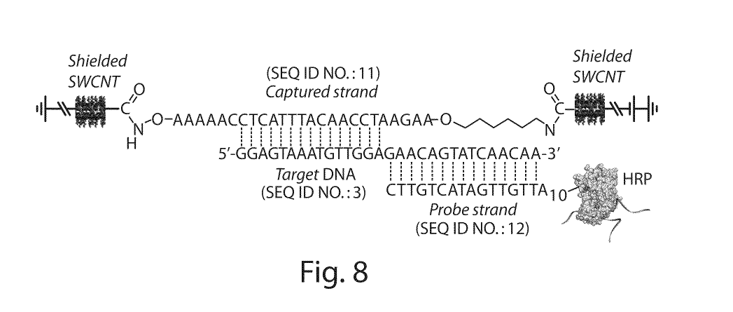

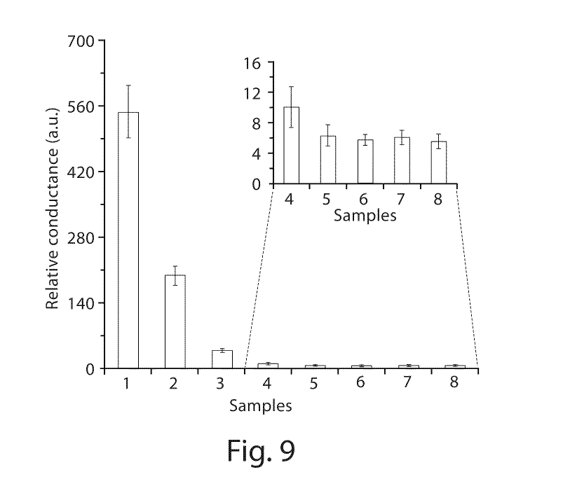

Studies to detect DNA, by means of simple conductivity measurements were performed using the scheme shown in FIG. 1A(ii) and the details of the recognition domain are shown in FIG. 8. DNA detection studies with HRP probes were conducted using a range of DNA analyte concentrations from 1 nM to 1 fM. Conductance changes of the devices after silver deposition were measured at a fixed development time of 2 min and are shown in FIG. 9. The relative conductance value for detection of the DNA analyte at nanomolar concentrations was 550 (20 MS) before silver development and 36.35 kΩ after silver development). The experiment was repeated with analyte concentrations ranging from 1 nM to 1 fM. FIG. 9 shows the conductimetric response data from concentration dependent DNA-detection studies, where the DNA concentrations for samples shown in Table 2. All experiments were conducted with a silver development time of 2 min. While no significant difference was found between control samples 5-8 in accordance with the t-test at the 95% confidence level, a significant difference was found between sample 4 and the controls (inset, FIG. 9). The error bars represent mean values±s.d.

TABLE 2
Concentration of DAN in Samples 1-8, as shown in FIG. 9.

| Sample | DNA Concentration | |
|---|---|---|
| 1 | 1 | nM |
| 2 | 10 | pM |
| 3 | 1 | pM |
| 4 | 10 | fM |
| 5 | 1 | fM |

| | |
|---|---|
| 6 | 1 nM control DNA |
| 7 | no DNA |
| 8 | no DNA/no probe |

The control experiment where complete mismatch DNA was used as a negative control did not lead to any significant silver deposition and resulted in a low conductivity change (FIG. 9). In addition, control experiments in the absence of analyte or DNA probe led to negligible conductivity increases. The relative conductance of the device was shown to decrease as a function of decreasing analyte concentration. The detection limit of DNA was found to be ˜10 fM.

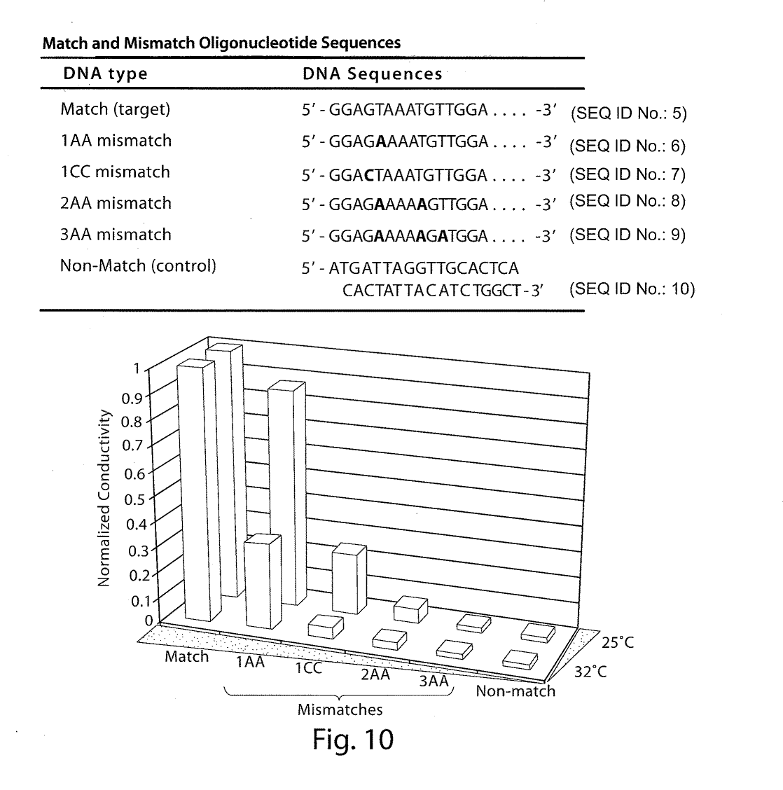

Specificity studies were conducted beyond the full mismatch control (sample 6, FIG. 10) to elucidate the impact of single, double, and triple base pair mismatches in the oligonucleotides analyte sequence. FIG. 10 shows the normalized conductimetric response data for single, double, and triple mismatch oligonucleotides sequences compared to the match sequence. The sensitivity of the response was shown to decrease as the number of mismatch base pairs increased. Additionally, there was a gain in sensitivity when the experiment was conducted at a slightly elevated temperature, at least partially due to an increase in oligonucleotide hybridization kinetics. For a single base pair mismatch, a ˜70% decrease in the response was observed, relative to the analyte containing the fully matched sequence at 32° C. When the experiment was conducted at 25° C., mismatch detection sensitivity significantly diminished relative to a matched analyte with <20% decrease in response.

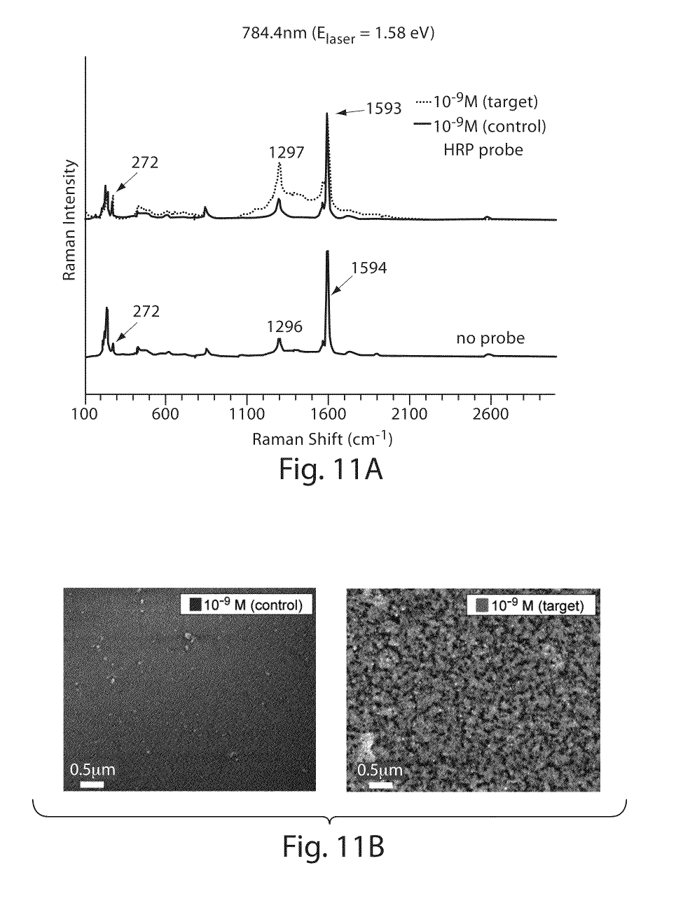

Additionally, SEM characterization of the device junctions showed silver deposition as a result of DNA detection. FIG. 11B shows SEM images of DNA-CNT nanowire devices. The left image shows SEM characterization of DNA-CNT nanowire device after mismatch DNA control experiment (1 nM conc.), showing a lack of silver deposition after development with HRP, while the right image shows SEM characterization of DNA-CNT nanowire device after target DNA detection experiment (1 nM conc.), showing silver deposition after development with HRP. (Scale bar in all micrographs=0.5 μm.)

In order to further characterize the sensing gap of the device before and after silver deposition, laser scanning Raman confocal microscopy was performed. Previous resonance Raman studies have demonstrated that SWCNTs filled with metallic silver lead to Fermi level alteration where silver behaves as an electron donor, which is consistent with an increase in electron carrier density. Utilizing laser scanning confocal

Raman microscopy, the device junction was characterized before and after analyte (1 nM) detection in addition to characterization of the control device. FIG. 11A shows confocal Raman data for the device junctions before (bottom curve=no probe) and after silver deposition (top curve=target device, middle curve=mismatch control device). The data was obtained using laser wavelength of 784.4 nm (Elaser=1.58 eV). All DNA analyte and control concentrations are shown at 1 nM and a ×50 microscope objective was used in all Raman experiments.

Silver modification of the nanotubes after analyte detection resulted in significant changes in the Raman spectra, as shown in FIG. 11. Changes in the radial breathing mode (RBM), D-band, G-band, and G′-band regions were observed. The RBM region showed three major peaks at 221, 230, and 238 cm⁻¹, which decreased in intensity after silver deposition. Additionally, an RBM peak at ˜270 cm⁻¹ showed a significant increase in intensity upon silver deposition consistent with increased tube-tube interactions. The intensity of the D-band increased with significant broadening upon silver deposition and shows a slight ˜1-2 cm⁻¹ shift to lower frequency from 1296 cm⁻¹, consistent with previous studies of SWCNTs on silver surfaces. Additionally, there was an increase in the D/G ratio upon silver deposition reflecting modification of the nanotube surface. The G+ (˜1593 cm⁻¹) and G− (˜1564 cm⁻¹) bands also showed a slight decrease in frequency and the ratio of the G−/G+ band intensity increased from 0.15 for the device without silver deposition to 0.31 with silver deposition, which is consistent with an increase in the metallic state of the nanotubes. Characterization of device junctions after mismatch DNA control (1 nM) treatment and development using HRP catalyzed silver deposition showed similar results to untreated devices, indicating a lack of silver deposition in the absence of analyte or presence of mismatch control. (FIG. 11)

The development of hybrid biocatalyzed-CNT network based detection methods can offer a highly sensitive and specific platform for the fabrication of simple and effective conductimetric devices. As shown in this example, peroxidase enzymes can provide a specific method for silver deposition at disrupted nanotube network connections, leading to amplified conductimetric detection of analytes.

### Example 11

The following example describes various methods used in the assay described in Example 10.

HRP crystal structure analysis and rendering. The crystal structure of horseradish peroxidase (PDB 1hch) was analyzed, rendered, and solvent accessible surface area calculations were performed using the UCSF Chimera software package.

HRP functionalization with DNA. 1.75 nmol of horseradish peroxidase (HRP) was reacted with 8.75 nmol of Sulfo-GMBS and 17.5 nmol thiolated DNA (Probe DNAs) in a ratio of 1:5:10, for 3 h at room temperature. The excess Sulfo-GMBS and DNA were removed using a Microcon centrifugal filter device unit (cut-off MW 30,000). Prior to modification, the disulfide bonds in all oligonucleotides were reduced by soaking in 0.1 M DTT in disulfide cleavage buffer for 2-3 h (100D of lyophilized DNA is typically reduced with 150 μL of freshly prepared solution of 0.1 M DTT). The deprotected DNA solutions were purified through desalting NAP-5 columns, and the amount of DNA from each column was determined by reading the absorbance of the solutions at 260 nm. The modified HRP was diluted to a final concentration of 4 μM with storage buffer and stored at −30° C.

Determination of number of DNA oligomers per HRP molecule. Concentrations of 5′-HRP-labeled oligonucleotides (probe DNA) were calculated either by the peak area at 260 nm (DNA concentration) and/or by incubating serial dilutions of the 5′-HRP oligonucleotides. Standard dilutions of native HRP were used for calibration at 402 nm. DNA-labeled HRP concentrations were interpolated from the calibration curve. Comparing the spectra of native HRP, free oligonucleotide, and oligonucleotide-modified HRP, the number of DNA strands conjugated per each HRP molecule was determined.

Enzymatic activity of the DNA-HRP conjugates compared to native HRP. The activity of the enzyme was determined colorimetrically using a UV-Vis spectrophotometer. A mixture of 8.7 mM ABTS, 0.01% hydrogen peroxide (wt/wt), 0.004% BSA (wt/v), 0.008% Triton X-100 (v/v) and 0.1 nM of the native HRP or the modified HRP, placed in PBS buffer, pH 7.4. The spectrophotometer was adjusted to 414 nm. Increase in absorbance was recorded for 5 min. The concentration of HRP was diluted so that a reasonable reaction rate (usually ΔA/min=0.02-0.04) could be obtained.

Device preparation. In a typical experiment, microelectrodes (50 nm Au on 10 nm Cr) with 250 μm gaps were prepared on glass substrates using a shadow mask. The exposed glass of the entire chip was treated with 0.2% OTS to block the surface Si—OH bonds as follows: the chips were cleaned with hot ethanol, dried with nitrogen, and cleaned for 30 seconds using a UV-Ozone cleaner. Next, the chips were soaked in a solution containing 0.2% OTS (v/v) in toluene for 15 minutes. The chips were washed with pure toluene to remove excess OTS, and dried using nitrogen followed by 30 minutes at 90° C. in an oven. Finally, the chips were soaked in toluene for 5 minutes with sonication using an ultrasonic bath. SWCNT-DNA nanowires (0.5 μL) were spin coated at 2,000 rpm between the two electrodes of each device, followed by a second 75 nm gold layer deposition on top of the nanowires. Chips were immersed in blocking buffer for 1 h to block the vacant gold surface with MCH and to block the vacant SWCNT surface with Tween 20. Finally, the chips were washed with NANOpure water to remove excess MCH and Tween 20, followed by drying with nitrogen.

DNA detection assay. The target DNA in assay buffer was reacted with the device surface followed by incubation at 25° C. for 2 h to allow hybridization between the captured DNA strands and the target DNA. Next, the devices were washed with the same assay buffer (5×1 mL). The corresponding probe was added to the capture DNA-target complexes, and hybridization was allowed to proceed at 25° C. for 1 h. The devices were washed with assay buffer (5×1 mL), washing buffer A (5×1 mL), washing buffer B (5×1 mL) and washing buffer C (5×1 mL) to remove nonspecifically bound enzyme probe and chloride ions that can react with the silver ions in the next step. Finally, the devices were soaked briefly in NANOpure water and dried with nitrogen. The conductance was recorded with a Fluke 287 multimeter before and after silver deposition. Silver deposition was accomplished using an EnzMet™ solution of reagents A, B and C in a 1:1:1 ratio for 2 minutes of incubation at room temperature.

Mismatch detection assay. The same procedure for the DNA detection assay was applied for mismatch detection. All DNA concentrations were fixed at 1 nM and in one set of experiments the temperature was changed to 32° C.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed:

1. A composition, comprising:
a nanostructure network comprising a plurality of nanostructures and a plurality of linkers attached to the nanostructures, such that the nanostructure species and linker species are arranged in a substantially continuous manner along the nanostructure network.

2. A composition as in claim 1, wherein the network of nanostructures comprises a linear network.

3. A composition as in claim 1, wherein the network of nanostructures comprises a branched network.

4. A composition as in claim 1, wherein the nanostructure species and linker species are arranged in an alternating manner along the nanostructure network.

5. A composition as in claim 1, wherein the nanostructures are attached to the linkers via sites at or near terminal ends of the nanostructures.

6. A composition as in claim 1, comprising:
a network of nanostructures comprising the formula,
-[A-B—]ₙ— or —[B-A-B]ₙ—,
wherein A is a nanostructure comprising at least two terminal ends;
B is a linker; and
n is at least 1,
wherein the linker is attached to the nanostructure at or near a terminal end of the nanostructure.

7. A composition as in claim 1, wherein the nanostructure species and linker species are attached via covalent bonds, non-covalent bonds, or DNA hybidization.

8-9. (canceled)

10. A composition as in claim 1, wherein the nanostructures comprise biological molecules positioned at or near terminal ends of the nanostructures.

11. A composition as in claim 10, wherein the biological molecule is a nucleic acid molecule, peptide, protein, glycoprotein, enzyme, DNAzyme, aptamer, hormone, antibody, antigen, cell, bacteria, virus, or carbohydrate.

12. A composition as in claim 1, wherein the nanostructures comprise single-strand DNA groups positioned at or near terminal ends of the nanostructures.

13. A composition as in claim 1, wherein the nanostructures are nanotubes, nanorods, nanoribbons, nanowires, or nanoparticles.

14. A composition as in claim 1, wherein the nanostructures are nanotubes.

15. A composition as in claim 1, wherein the nanostructures are conductive nanotubes, semiconductive nanotubes, or metallic nanotubes.

16. A composition as in claim 1, wherein the nanostructures are single-walled carbon nanotubes or multi-walled carbon nanotubes.

17. A composition as in claim 1, wherein the nanostructures are metal nanowires.

18. A composition as in claim 1, wherein the linker comprises a biological molecule.

19. A composition as in claim 1, wherein the linker comprises a nucleic acid molecule, peptide, protein, glycoprotein, enzyme, DNAzyme, aptamer, hormone, antibody, antigen, cell, bacteria, virus, or carbohydrate.

20. A composition as in claim 1, wherein the linker comprises DNA.

21. A composition as in claim 1, wherein the linker comprises a polymer.

22. A composition as in claim 1, wherein the linker comprises a conducting polymer.

23. A composition as in claim 1, wherein the linker comprises a nanoparticle.

24. A composition as in claim 1, wherein the linker comprises a metal nanoparticle.

25. A composition as in claim 1, wherein the linker is substituted with at least one biological molecule.

26. A composition as in claim 1, wherein the linker comprises single-strand DNA groups.

27. A composition as in claim 1, wherein the nanostructures comprise a first set of single-strand DNA groups positioned at or near terminal ends of the nanostructures, and the linkers comprise a second set of single-strand DNA groups, wherein the first set of single-strand DNA groups is complementary to the second set of single-strand DNA groups.

28. A composition as in claim 1, wherein the network of nanostructures further comprises at least one site capable of interacting with an analyte.

29. A sensor, comprising:
a composition as in claim 1.

30. A sensor as in claim 29, wherein the sensor further comprises a source of external energy applicable to the composition to generate a determinable signal; and
a detector positioned to detect the signal.

31. A device, comprising:
a composition as in claim 1;
at least two electrodes in electrochemical communication with the composition; and
optionally, a conductive material in electrochemical communication with the composition and at least two electrodes.

32. A method for determining an analyte, comprising:
exposing, to an environment suspected of containing an analyte, a device comprising a network of nanostructures positioned relative to each other so as to define an area between adjacent nanostructures, wherein the analyte, if present, interacts with at least a portion of the device to produce a species proximate the area between adjacent nanostructures that affects a property of interaction between the nanostructures, thereby producing a detectable change in a property of the network of nanostructures, whereby the detectable change in the property of the network can be determined to determine the analyte.

33. A method for determining an analyte, comprising:
exposing, to an environment suspected of containing an analyte, a network of nanostructures positioned relative to each other so as to define an area between adjacent nanostructures, wherein the analyte, if present, interacts with at least a portion of the device to produce a conductive species proximate the area between adjacent nanostructures, thereby generating a determinable signal; and
determining the signal.

34-113. (canceled)
